SEQUENCE LISTING (28 sequences)

SEQ ID NO: 1 - DNA, 30 bp, Artificial Sequence
  Description of Artificial Sequence frizzled-related protein (FRZB) wild-type allele-specific PCR amplification Forward primer
ctggcaggaactcgaacccccggcaagcac

SEQ ID NO: 2 - DNA, 30 bp, Artificial Sequence
  Description of Artificial Sequence frizzled-related protein (FRZB) wild-type allele-specific PCR amplification Reverse primer
cttaagagtctgcccccaaaccattacaaa

SEQ ID NO: 3 - DNA, 30 bp, Artificial Sequence
  Description of Artificial Sequence frizzled-related protein (FRZB) mutant 1178/G allele-specific PCR amplification Forward primer
gttagaatcatggaaataatgaccctggtg

SEQ ID NO: 4 - DNA, 30 bp, Artificial Sequence
  Description of Artificial Sequence frizzled-related protein (FRZB) mutant 1178/G allele-specific PCR amplification Reverse primer
ttactttttgtatttcgggatttagttggc

SEQ ID NO: 5 - DNA, 45 bp, Artificial Sequence
  Description of Artificial Sequence frizzled-related protein (FRZB) exon 4 single nucleotide polymorphism (SNP) wild-type position 806 C
aacaattacaactatgtcattcgggctaaagttaaagagataaag

SEQ ID NO: 6 - protein, 15 aa, Artificial Sequence
  Description of Artificial Sequence frizzled-related protein (FRZB) exon 4 single nucleotide polymorphism (SNP) wild-type residue 200 Arg
NNYNYVIRAKVKEIK

SEQ ID NO: 7 - DNA, 45 bp, Artificial Sequence
  Description of Artificial Sequence frizzled-related protein (FRZB) exon 4 single nucleotide polymorphism (SNP) mutant position 806 T
aacaattacaactatgtcatttgggctaaagttaaagagataaag

SEQ ID NO: 8 - protein, 15 aa, Artificial Sequence
  Description of Artificial Sequence frizzled-related protein (FRZB) exon 4 single nucleotide polymorphism (SNP) mutant residue 200 Trp
NNYNYVIWAKVKEIK

SEQ ID NO: 9 - DNA, 39 bp, Artificial Sequence
  Description of Artificial Sequence frizzled-related protein (FRZB) exon 6 single nucleotide polymorphism (SNP) wild-type position 1178 C
aagtctggcaggaactcgaacccccggcaagcacgcaac

SEQ ID NO: 10 - protein, 13 aa, Artificial Sequence
  Description of Artificial Sequence frizzled-related protein (FRZB) exon 6 single nucleotide polymorphism (SNP) wild-type residue 324 Arg
KSGRNSNPRQARN

SEQ ID NO: 11 - DNA, 39 bp, Artificial Sequence
  Description of Artificial Sequence frizzled-related protein (FRZB) exon 6 single nucleotide polymorphism (SNP) mutant position 1178 G
aagtctggcaggaactcgaacccccggcaagcaggcaac

SEQ ID NO: 12 - protein, 13 aa, Artificial Sequence
  Description of Artificial Sequence frizzled-related protein (FRZB) exon 6 single nucleotide polymorphism (SNP) mutant residue 324 Gly
KSGRNSNPRQAGN

SEQ ID NO: 13 - DNA, 1476 bp, Homo sapiens
  frizzled-related protein (FRZB) cDNA
acggggcctgggcggsaggggcggtggctggagctcggtaaagctcgtgggaccccattgggggaatttgatccaaggaagcggtgattgccgggggaggagaagctcccagatccttgtgtccacttgcagcgggggaggcggagacgcggagcgggccttttggcgtccactgcgcggctgcaccctgccccatcctgccgggatcatggtctgcggcagcccgggagggatgctgctgctgcgggccgggctgcttgccctggctgctctctgcctgctccgggtgcccggggctcgggctgcagcctgtgagcccgtccgcatccccctgtgcaagtccctgccctggaacatgactaagatgcccaaccacctgcaccacagcactcaggccaacgccatcctggccatcgagcagttcgaaggtctgctgggcacccactgcagccccgatctgctcttcttcctctgtgccatgtacgcgcccatctgcaccattgacttccagcacgagcccatcaacccctgtaagtctgtgtgcgagcgggcccggcagggctgtgagcccatactcatcaagtaccgccactcgtggccggagaacctggcctgcgaggagctgccagtgtacgacaggggcgtgtgcatctctcccgaggccatcgttactgcggacggagctgattttcctatggattctagtaacggaaactgtagaggggcaagcagtgaacgctgtaaatgtaagcctattagagctacacagaagacctatttccggaacaattacaactatgtcattcgggctaaagttaaagagataaagactaagtgccatgatgtgactgcagtagtggaggtgaaggagattctaaagtcctctctggtaaacattccacgggacactgtcaacctctataccagctctggctgcctctgccctccacttaatgttaatgaggaatatatcatcatgggctatgaagatgaggaacgttccagattactcttggtggaaggctctatagctgagaagtggaaggatcgactcggtaaaaaagttaagcgctgggatatgaagcttcgtcatcttggactcagtaaaagtgattctagcaatagtgattccactcagagtcagaagtctggcaggaactcgaacccccggcaagcacgcaactaaatcccgaaatacaaaaagtaacacagtggacttcctattaagacttacttgcattgctggactagcaaaggaaaattgcactattgcacatcatattctattgtttactataaaaatcatgtgataactgattattacttctgtttctcttttggtttctgcttctctcttctctcaacccctttgtaatggtttgggggcagactcttaagtatattgtgagttttctatttcactaatcatgagaaaaactgttcttttgcaataataataaattaaacatgctgtta

SEQ ID NO: 14 - protein, 325 aa, Homo sapiens
  frizzled-related protein (FRZB, Fritz, Frzb-1, fre, frizzled (Drosphila) homolog-related, fzrb, hfiz)
MVCGSPGGMLLLRAGLLALAALCLLRVPGARAAACEPVRIPLCKSLPWNMTKMPNHLHHSTQANAILAIEQFEGLLGTHCSPDLLFFLCAMYAPICTIDFQHEPINPCKSVCERARQGCEPILIKYRHSWPENLACEELPVYDRGVCISPEAIVTADGADFPMDSSNGNCRGASSERCKCKPIRATQKTYFRNNYNYVIRAKVKEIKTKCHDVTAVVEVKEILKSSLVNIPRDTVNLYTSSGCLCPPLNVNEEYIIMGYEDEERSRLLLVEGSIAEKWKDRLGKKVKRWDMKLRHLGLSKSDSSNSDSTQSQKSGRNSNPRQARN

SEQ ID NO: 15 - DNA, 19 bp, Artificial Sequence
  Description of Artificial Sequence frizzled-related protein (FRZB) 5′ untranslated region (UTR) PCR amplification Forward primer
gggaggtaggaaagtgcag

SEQ ID NO: 16 - DNA, 20 bp, Artificial Sequence
  Description of Artificial Sequence frizzled-related protein (FRZB) 5′ untranslated region (UTR) PCR amplification Reverse primer
ggtggttgggcatcttagtc

SEQ ID NO: 17 - DNA, 20 bp, Artificial Sequence
  Description of Artificial Sequence frizzled-related protein (FRZB) exon 1 PCR amplification Forward primer
gaggagaagctcccagatcc

SEQ ID NO: 18 - DNA, 20 bp, Artificial Sequence
  Description of Artificial Sequence frizzled-related protein (FRZB) exon 1 PCR amplification Reverse primer
ccaagaattgaggaggctgt

SEQ ID NO: 19 - DNA, 20 bp, Artificial Sequence
  Description of Artificial Sequence frizzled-related protein (FRZB) exon 2 PCR amplification Forward primer
gccgacctcatgacgttaat

SEQ ID NO: 20 - DNA, 20 bp, Artificial Sequence
  Description of Artificial Sequence frizzled-related protein (FRZB) exon 2 PCR amplification Reverse primer
gaccacaaatgaaaaccagg

SEQ ID NO: 21 - DNA, 20 bp, Artificial Sequence
  Description of Artificial Sequence frizzled-related protein (FRZB) exon 3 PCR amplification Forward primer
aagagccttctcaccaccaa

SEQ ID NO: 22 - DNA, 21 bp, Artificial Sequence
  Description of Artificial Sequence frizzled-related protein (FRZB) exon 3 PCR amplification Reverse primer
aaatggctccactcgtttaca

SEQ ID NO: 23 - DNA, 22 bp, Artificial Sequence
  Description of Artificial Sequence frizzled-related protein (FRZB) exon 4 PCR amplification Forward primer
ccattcgttgaatatcaagtgg

SEQ ID NO: 24 - DNA, 20 bp, Artificial Sequence
  Description of Artificial Sequence frizzled-related protein (FRZB) exon 4 PCR amplification Reverse primer
aatgcgagggtagcatctct

SEQ ID NO: 25 - DNA, 24 bp, Artificial Sequence
  Description of Artificial Sequence frizzled-related protein (FRZB) exon 5 PCR amplification Forward primer
ttcttgttcaagtactgtttcctt

SEQ ID NO: 26 - DNA, 20 bp, Artificial Sequence
  Description of Artificial Sequence frizzled-related protein (FRZB) exon 5 PCR amplification Reverse primer
ttcaatgacagatggctggt

SEQ ID NO: 27 - DNA, 21 bp, Artificial Sequence
  Description of Artificial Sequence frizzled-related protein (FRZB) exon 6/3′ untranslated region (UTR) PCR amplification Forward primer
cctggtgatatgtgcttgtga

SEQ ID NO: 28 - DNA, 21 bp, Artificial Sequence
  Description of Artificial Sequence frizzled-related protein (FRZB) exon 6/3′ untranslated region (UTR) PCR amplification Reverse primer
caattggggtgcagaaagtaa

(19) United States
(12) Patent Application Publication
(10) Pub. No.: US 2005/0130199 A1
(43) Pub. Date: Jun. 16, 2005

(54) Mutations in WNT-frizzled signaling pathways associated with osteoarthritis

(75) Inventors: Dennis A. Carson, La Jolla, CA (US); Mary P. Corr, San Diego, CA (US); John Loughlin, Headington (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA (US)

(21) Appl. No.: 10/947,849

(22) Filed: Sep. 22, 2004

Related U.S. Application Data
(60) Provisional application No. 60/505,122, filed on Sep. 22, 2003.

(51) Int. Cl. C12Q 1/68; G01N 33/53; G01N 33/567; C07H 21/04

(52) U.S. Cl. 435/6; 514/44; 435/7.2; 536/24.3

(57) ABSTRACT

This invention provides methods to identify individuals predisposed to developing osteoarthritis, to diagnose osteoarthritis, and to monitor the progression of the disease. The method also provides methods to identify modulators of bone development that affect a wnt/fzd signaling pathway and methods to prevent or treat osteoarthritis by administering the modulator of bone development.

### CROSS-REFERENCES TO RELATED APPLICATIONS

This application claims the benefit of U.S. Provisional Application No. 60/505,122, filed Sep. 22, 2003, which is herein incorporated by reference for all purposes.

### STATEMENT AS TO RIGHTS TO INVENTIONS MADE UNDER FEDERALLY SPONSORED RESEARCH OR DEVELOPMENT

This invention was made with Government support of Grant No. AR50901 awarded by NIH. The Government has certain rights in this invention.

### FIELD OF INVENTION

This invention provides methods to identify individuals predisposed to developing osteoarthritis, to diagnose osteoarthritis, and to monitor the progression of the disease. The method also provides methods to identify modulators of bone development that affect a wnt/fzd signaling pathway and methods to prevent or treat osteoarthritis by administering the modulator of bone development.

### BACKGROUND OF THE INVENTION

Osteoarthritis (OA), “is a condition of synovial joints characterized by focal cartilage loss and an accompanying reparative bone response” (Jones, A., and M. Doherty, Bmj, 310:457-460 (1995)). However, OA is clearly a heterogeneous disease. Separate risk factors may predispose to OA in large and small joints, and may distinguish between men and women (Radin, E. L., et al., Lancet, 1:519-522 (1972); Jones, G., et al., J Rheumatol, 29:1719-1724 (2002); Schneider, D. L., et al., J Rheumatol, 29:1467-1472 (2002); Hart, D. J., et al., Arthritis Rheum, 42:17-24 (1999); Hart, D. J., and T. D. Spector, Ann Rheum Dis., 52:93-96 (1993); Arden, N. K., et al., Arthritis Rheum, 42:1378-1385 (1999); Sowers, M., L., et al., Arthritis Rheum, 42:483-489 (1999); Hochberg, M. C., et al., J Rheumatol, 22:2291-2294 (1995)). For example, recent studies showed that a higher bone density increases the risk of incident radiographic knee OA in older women, but this relationship has not been seen to the same degree in men, nor in OA of the hands (Sowers, M., L., et al., Arthritis Rheum, 42:483-489 (1999); Hannan, M. T., et al., Arthritis Rheum, 36:1671-1680 (1993) Hart, D. J., et al., Ann Rheum Dis., 53:158-162 (1994); Burger, H., et al., Arthritis Rheum, 39:81-86 (1996); Zhang, Y., et al., J Rheumatol, 27:1032-1037 (2000); Lane, N. E., and M. C. Nevitt, J Rheumatol, 21:1393-1396 (1994); Lane, N. E., et al., J Bone Miner Res., 10:257-263 (1995)). Nonetheless, experiments of this type have led to the suggestion that, in some cases, articular cartilage may be the innocent bystander of a disease process that initiates more in subchondral bone than in cartilage (Dieppe, P., Osteoarthritis Cartilage, 7:325-326 (1999); Dieppe, P., Bmj., 318:1299-1300 (1999)).

Common biochemical changes observed in osteoarthritis (OA) include the enhanced turnover of cartilage and bone matrix components, and the upregulation of gene products synthesized in immature cartilage and bone. However, no single anomaly has yet been shown to be common to all patients, nor to be present early in the disease when interventional therapy may be of the greatest benefit. Thus, a major goal of OA research is to identify biomarkers that reflect the genetic epidemiology of OA, and that potentially could lead to new therapeutic targets. The present invention solves these and other problems.

### BRIEF SUMMARY OF THE INVENTION

In one embodiment, the present invention provides a method to determine a predisposition to osteoarthritis in a patient, by obtaining biological sample from the patient and detecting a mutation in wnt/fzd pathway member nucleic acid when compared to a control. Wnt/fzd pathway members include, for example, nucleic acids that encode proteins involved in wnt/fzd signaling, e.g., a wnt protein, a fzd protein, an SFRP, an LRP, a DKK protein, a WIF protein, or a WISP protein. In one aspect, the mutation results in increased signaling in a wnt/fzd pathway.

In a preferred embodiment, the wnt/fzd pathway member nucleic acid is a FRZB nucleic acid that encodes the FRZB protein. The mutation is detected by determining the sequence of the FRZB gene of the patient, e.g., using PCR based methods, hybridization based methods, or direct sequencing of the gene or a PCR product derived from the gene. In one embodiment, the FRZB nucleic acid encodes a tryptophan at residue 200, rather than the arginine residue found in a human population that does not have a predisposition to OA, i.e., the control. In one aspect, the tryptophan at residue 200 of the FRZB protein is the result of a C to T change at base 806 in the FRZB nucleic acid. In another embodiment, the FRZB nucleic acid encodes a glycine at residue 324, rather than the arginine residue found in a human population that lacks a predisposition to OA, i.e., the control. In one aspect, the glycine at residue 324 of the FRZB protein is the result of a C to G change at base 1178 in the FRZB nucleic acid. In a further embodiment, the FRZB protein has both a tryptophan at residue 200 and a glycine at residue 324. The double mutation is the result of a C to T change at residue 806 and a C to G change at residue 1178.

In one aspect, the mutation indicates a predisposition to a change in bone mass or bone structure at a region adjacent to a joint, for example a knee or hip joint. The change in bone mass or bone structure can be detected using X-ray, magnetic resonance imaging (MRI), or ultrasound technology.

In another aspect the patient is an adult and in a further aspect the patient is female.

In another embodiment the invention provides a method to determine a predisposition to osteoarthritis in a patient, by obtaining biological sample from the patient and detecting a difference in a wnt/fzd pathway member protein expression when compared to a control, i.e. a human population that does not have a predisposition to OA. In a preferred embodiment the biological sample is a serum sample. In another embodiment, the difference in wnt/fzd pathway member protein expression indicates increased signaling in a wnt/fzd pathway.

The difference in protein expression can be a difference in a single wnt/fzd pathway member protein, or a difference in expression of more than one wnt/fzd pathway member proteins. In one embodiment the difference in wnt/fzd pathway member protein expression is detected using mass spectroscopy (MS). In one embodiment the difference in wnt/fzd pathway member protein expression is detected using immunoassays, i.e. assays based on antibodies specific for the wnt/fzd pathway member protein(s).

In one aspect the difference in wnt/fzd pathway member protein expression indicates a predisposition to a change in bone mass or bone structure at a region adjacent to a joint. The change in bone mass or bone structure can be detected using X-ray, magnetic resonance imaging (MRI), or ultrasound technology.

In another aspect the patient is an adult and in a further aspect the patient is female.

In a further embodiment, the present invention provides a method of identifying a compound that modulates bone formation, the method comprising the steps of contacting a chondrocyte comprising a wnt/fzd pathway member protein or fragment thereof and determining the functional effect of the compound upon a bone formation assay, thereby identifying a compound that modulates bone formation. Compounds identified as bone formation modulators can be administered to a patient used to modulating bone formation in a subject, e.g., to treat osteoarthritis or to prevent or slow development of osteoarthritis.

In another aspect, the present invention also provides PCR primers for detecting the sequence of a wild-type FRZB nucleic acid, the primer pair consisting of a first primer comprising CTggCAggAACTCgAACCCCCggCAAgCAC, and a second primer comprising CTTAAgAGTCTgCCCCCAAACCATTACAAA. In a further aspect, the present invention provides PCR primers for detecting the sequence of a mutant FRZB nucleic acid, the primer pair consisting of a first primer comprising gTTAgAATCATggAAATAATgACCCTggTg and a second primer comprising TTACTTTgTATTTCgggATTTAgTTggC.

### BRIEF DESCRIPTION OF THE DRAWINGS

FIG. 1. FIG. 1 depicts FRZB (sFRP3) expression in human chondrocytes. Chondrocyte lines (lanes 1 and 2) and synovial fibroblast-like cells (lanes 3 and 4) in culture were used. The cells were lysed in RIPA buffer and thirty micrograms of protein in each lane was separated by SDS-PAGE. After transfer to PDVF, the membrane was serially probed with the indicated antibodies (anti-SFRP-3, R&D Systems; anti-SFRP-1 and 2, Santa Cruz Biotechnology Inc.).

FIG. 2. FIG. 2 depicts the domain structure of FRZB and the SNPs identified in exons 4 and 6.

FIG. 3. FIG. 3 depicts determination of allele specific amplicons in FZRB. Lane 1 is Jurkat DNA, Lane 2-4 are population pools of genomic DNA, and Lane 5 is control DNA for the mutant allele.

FIG. 4. FIG. 4 depicts the ability of wild type FRZB, and the 806 (arg-trp) and 1178 (arg-gly) variants to antagonize wnt-signaling after transient transfection into HEK293 cells. HEK293 cells were transfected with the TOPflash reporter, a β-galactosidase and a wnt-1 expressing constructs and the indicated FRZB genes cloned into pcDNA3. The ordinate uses arbitrary units to show the average relative activities of the wnt-dependent TOPFLASH reporter gene+SEM. The luciferase activities were normalized to β-galactosidase activity to control for transfection efficiency. The FRZB vectors had no effect on the control FOPFLASH reporter gene (not shown). The transfections were performed in duplicate and the luciferase assay was performed in duplicate.

FIG. 5 provides a nucleic acid sequence (top) and the encoded amino acid sequence (bottom) for human FRZB.

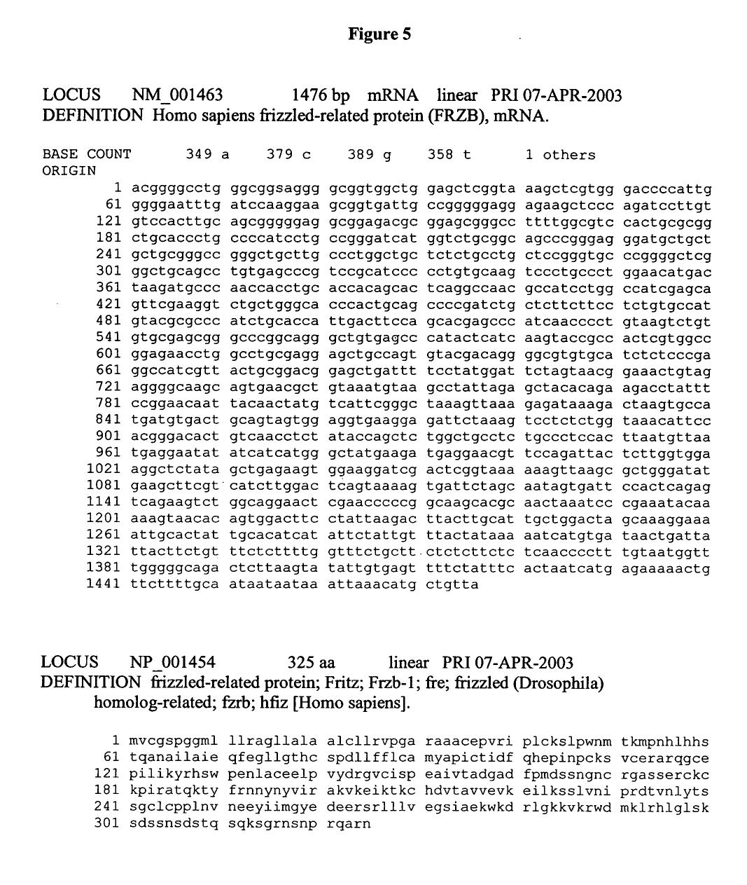

### Definitions

The terms “Wnt protein” or “Wnt ligand” refer to a family of mammalian proteins related to the Drosophila segment polarity gene, wingless. In humans, the Wnt family of genes typically encode 38 to 43 kDa cysteine rich glycoproteins having hydrophobic signal sequence, and a conserved asparagine-linked oligosaccharide consensus sequence (see e.g., Shimizu et al Cell Growth Differ 8:1349-1358 (1997)). The Wnt family contains at least 16 mammalian members. Exemplary Wnt proteins include Wnt-1, Wnt-2, Wnt-3, Wnt-3A, Wnt-4, Wnt-5A, Wnt-6, Wnt-7A, Wnt-7B, Wnt-8A, Wnt-8B, Wnt-10B, Wnt-11, Wnt-13, Wnt 14, Wnt 15, and Wnt 16. Exemplary amino acid and nucleic acid sequences for Wnt proteins are disclosed in U.S. patent application Ser. No. 10/285,976; filed Nov. 1, 2002; which is herein incorporated by reference for all purposes.

The terms “frizzled protein” or “frizzled receptor” or “fzd” refer to a family of mammalian proteins related to the Drosophila frizzled genes, which play a role in the development of tissue polarity. The Frizzled family comprises at least 10 mammalian genes. Exemplary human Frizzled receptors include Frizzled1, Frizzled2, Frizzled3, Frizzled4, Frizzled5, Frizzled6, Frizzled7, Frizzled8, Frizzled9 and Frizzled10. Exemplary amino acid and nucleic acid sequences for Fzd proteins are disclosed in U.S. patent application Ser. No. 10/285,976; filed Nov. 1, 2002; which is herein incorporated by reference for all purposes. The mammalian homologues of the Drosophila frizzled protein share a number of common structural motifs. The N terminus located at the extracellular membrane surface is followed by a signal sequence, a domain of 120 amino acids with an invariant pattern of 10 cysteine residues, and a highly divergent region of 40-100 largely variable hydrophilic amino acids. Putative hydrophobic segments form seven membrane-spanning helices linked by hydrophilic loops, ending with the C terminus located at the intracellular face of the membrane. The cysteine-rich domains (CRDs) and the transmembrane segments are strongly conserved, suggesting a working model in which an extracellular CRD is tethered by a variable linker region to a bundle of seven membrane-spanning-helices. Frizzled protein receptors are, therefore, involved in a dynamic model of transmembrane signal transduction analogous to G-protein-coupled receptors with amino-terminal ligand binding domains.

In addition to the Wnt ligands, a family of secreted frizzled-related proteins (SFRPs) has been isolated. SFRPs are also know as FRPs, SARPs, and FRZB proteins. SFRPs appear to function as soluble endogenous modulators of Wnt signaling by competing with the membrane-spanning frizzled receptors for the binding of secreted Wnt ligands. SFRPs, therefore, can modulate bone formation by exerting an antagonistic effect wnt/fzd signaling. SFRPs antagonize Wnt function by binding the protein and blocking access to its cell surface signaling receptor. Thus, decreasing the ability of SFRPs to bind to Wnt proteins can activate Wnt/Fzd signaling. Exemplary mammalian SFRPs include SFRP1 (also known as FRP or SARP2; nucleic acid accession number NM_{—}003012, amino acid accession number NP_{—}003003), SFRP2 (also known as SARP1; nucleic acid accession number XM_{—}050625, amino acid accession number XP_{—}050625), SFRP3 (also know as FRZB1 or FRZB; nucleic acid accession number NM_{—}001463, amino acid accession number NP_{—}001454), SFRP4 (also known as FRPHE or FRZB2; nucleic acid accession number NM_{—}003014, amino acid accession number NP_{—}003005), and SFRP5 (also known as SARP3; nucleic acid accession number NM_{—}003015, amino acid accession number NP_{—}003006). See, e.g., Melkonyan et al., PNAS USA 94:13636-13641 (1997); Hoang et al., J. Biol. Chem. 271:26131-26137 (1996); and Abu-Jawdeh et al., Lab. Invest. 79:439-447 (1999). Other proteins encompassed by the term SFRP include Dickkopf (DKK) proteins, e.g., DKK1 (nucleic acid accession number NM_{—}012242, amino acid accession number NP_{—}036374), DKK2 (nucleic acid accession number NM 014421, amino acid accession number NP_{—}055236), DKK3 (also known as Soggy; nucleic acid accession number NM_{—}013253, amino acid accession number NP_{—}037385), and DKK4 (nucleic acid accession number NM_{—}014420, amino acid accession number NP_{—}055235). DKK proteins are secreted proteins and are believed to block Wnt induced activation of Wnt/Fzd signaling pathways. See, e.g., Fedi, et al., J. Biol. Chem. 274:19465-19472 (1999) and Krupnik et al., Gene 283:301-313 (1999).

Other proteins involved in wnt/fzd signaling include LDL receptor related proteins (LRPs). The LRP5 and LRP6 proteins have been shown to bind to, and to enhance, signaling of wnt family proteins through their cognate frizzled (Fzd) receptors (Tamai, K., et al., Nature, 407:530-535 (2000); Wehrli, M., et al., Nature, 407:527-530 (2000)). Wnt Inhibitory Factors (WIFs) are secreted proteins that binds to and inhibit WNT proteins activities. WIF proteins contain a WNT inhibitory factor (WIF) domain and epidermal growth factor (EGF)-like domains. WIF proteins include WIF1. (See, e.g., Hsieh et al., Nature 398:431-436 (1999)). WNT inducible signaling pathway (WISP) proteins belongs to the connective tissue growth factor (CTGF) family. WISP proteins are downstream of WNTs in the WNT1 signaling pathway. WISP proteins include WISP1, WISP2, and WISP3. (See, e.g., Pennica et al., PNAS USA 95:14717-14722 (1998)).

The terms “wnt signaling”, “wnt/fzd signaling”, and “fzd signaling” are used interchangeably.

A “Wnt/Fzd signaling pathway” or “Wnt/Fzd pathway” refers to activation of an intracellular signal transduction pathway that is initiated by an interaction between a specific Wnt protein and a specific Fzd protein. Generally, the Wnt/Fzd interaction will be binding of a Wnt protein to a Fzd receptor, leading to activation of a signal transduction pathway. Wnt/fzd signaling pathway includes includes the activities of SFRPs, DKKs, LRPs, WIFs, and WISPs. In some instances activation of the Wnt/Fzd signaling pathway will lead to induction of downstream wnt and/or fzd inducible genes. A “downstream wnt/fzd regulated gene product” is a protein or RNA that is upregulated, or otherwise regulated, as a result of signaling by a wnt/fzd transduction pathway. Regulation of expression levels and activity levels are included.

“Wnt/Fzd signaling pathway member” or “Wnt/Fzd pathway member” refers to a nucleic acid that encodes a protein involved in wnt/fzd signaling, e.g., a wnt protein, a fzd protein, an SFRP, an LRP, a DKK protein, a WIF protein, or a WISP protein. “Wnt/Fzd signaling pathway member” or “Wnt/Fzd pathway member” also include proteins or polypeptides involved in wnt/fzd signaling, e.g., a wnt protein, a fzd protein, an SFRP, an LRP, a DKK protein, a WIF protein, or a WISP protein. Examples of wnt/fzd pathway member nucleic acids and proteins are found in.

Fragments are included in the definition of Wnt/Fzd signaling pathway member proteins. Portion, or fragment, in this sense includes sequences from at least 2 amino acids up to the full length sequence of a Wnt/Fzd signaling pathway member, e.g., a fzd protein or an LRP, minus one amino acid at either the N- or C-terminus.

The term “modulator” or grammatical equivalents as used herein describes any molecule, either naturally occurring or synthetic, e.g., protein (for example an antibody or Wnt), oligopeptide (e.g., from about 5 to about 25 amino acids in length, preferably from about 10 to 20 or 12 to 18 amino acids in length, preferably 12, 15, or 18 amino acids in length), small chemical molecule, polysaccharide, lipid (e.g., a sphingolipid), fatty acid, polynucleotide, oligonucleotide, etc., that directly or indirectly activates a Wnt/Fzd signaling pathway. Modulators include antagonists or inhibitors of a Wnt/Fzd signaling pathway, as well as agonists or activators of a Wnt/Fzd signaling pathway.

The terms “antagonists” or “inhibitors” of Wnt/Fzd signaling refer to compounds that, e.g., ininhibit or decrease Wnt signaling as measured in known assays for Wnt signaling (e.g., measurement of transcription of reporter genes, measurement of β catenin levels, or oncogene expression controlled by Tcf and Lef transcription factors, or decrease apoptosis). In one embodiment, the antagonist of Wnt/Fzd signaling binds directly to a Fzd receptor or to a Wnt protein. In another embodiment, the antagonist of Wnt/Fzd signaling is an SFRP protein or fragment thereof. In another embodiment, the antagonist of Wnt/Fzd signaling binds directly to an LRP protein. Inhibitors include modified versions of Wnt, Fzd, LRP, or SFRP proteins, as well as naturally occurring and synthetic ligands, antagonists, antibodies, small chemical molecules, and the like. Assays for detecting inhibitors of the invention are described in more detail below.

The terms “agonists” or “activators” of Wnt/Fzd signaling refer to compounds that, e.g., induce or increase Wnt signaling as measured in known assays for Wnt signaling (e.g., measurement of transcription of reporter genes, measurement of β catenin levels, or oncogene expression controlled by Tcf and Lef transcription factors, or decrease apoptosis). In one embodiment, the agonist of Wnt/Fzd signaling binds directly to a Fzd receptor. In another embodiment, the agonist of Wnt/Fzd signaling is a Wnt protein or fragment thereof. In another embodiment, the agonist of Wnt/Fzd signaling binds directly to an SFRP protein. Activators, include modified versions of Wnt, Fzd, or SFRP proteins, as well as naturally occurring and synthetic ligands, agonists, antibodies, small chemical molecules, and the like. Assays for detecting activators of the invention are described in more detail below.

A “cell that overexpresses a Wnt/Fzd pathway member” is a cell in which expression of a particular Wnt/Fzd pathway member protein is at least about 2 times, usually at least about 5 times the level of expression in a normal, cell from the same tissue (i.e., from a tissue that is not affected by osteoarthritis). In addition, expression of particular Wnt and/or Fzd and/or LRP and/or SFRP proteins can be compared to other Wnt and/or Frizzled and/or LRP, and/or SFRP proteins in the same cell. Such methods include RT-PCR, use of antibodies against the gene products, and the like.

“Osteoarthritis” refers to a degenerative disease of the joints caused by loss of cartilage. One of the hallmarks of osteoarthritis is increased apoptosis. See, e.g., Lotz et al., Osteoarthritis and Cartilage 7:389-391 (1999). Symptoms of osteoarthritis include joint pain and stiffness. One indicator of OA is a change in bone mass or structure at a regions adjacent to a joint. Changes include any of the following nonuniform joint space loss, osteophyte formation, cyst formation and subchondral sclerosis. Such changes can be determined using e.g., X-rays, magnetic resonance imaging, and ultrasound. A method of treating osteoarthritis refers to a reduction or elimination of the symptoms of osteoarthritis, or a reduction or elimination of change in bone mass or structure at a regions adjacent to a joint affected by osteoarthritis. A method of treating also includes slowing or halting the progression of the disease. In a preferred embodiment, the changes in bone formation occur in a region adjacent to a knee or hip joint.

A “predisposition to develop osteoarthritis” refers to an increased likelihood of an individual to develop osteoarthritis. The present invention presents biomarkers that are useful to determine whether an individual has an predisposition to develop OA, e.g., specific mutations in the FRZB gene and serum proteins that indicate a predisposition to the disease. Some other factors that render an individual susceptible to developing osteoarthritis (or grammatical equivalents thereof) are known. Those factors include aging, obesity, and previous joint injury.

Bone formation refers to changes in bone synthesis and resorption and includes remodelling of existing bone. Modulation of bone formation refers to changes in bone synthesis and resorption. In a preferred embodiment, changes in a wnt/fzd signaling pathway result in modulation of bone formation. Thus, modulators of a Wnt/Fzd signaling pathway, preferably modulate bone formation.

“Chondrocyte” refers to cells found in cartilage that secrete collagens and glucosaminoglycan. “Chondrocyte” also includes cells or cell lines derived from a chondrocyte. “Osteoblast” refers to cells found in the osteogenetic layer of the periosteum, and from or around which the matrix of the bone is developed. “Osteoblast” also includes cells or cell lines derived from an osteoblast. “Osteoclast” refers to cells that actively reabsorb bone. “Osteoclast” also includes cells or cell lines derived from an osteoclast. In some embodiments, chondrocytes, osteoblasts, and osteoclasts are used to identify compounds that modulate bone formation.

As used herein, “proteoglycan” refers to components of the extracellular matrix composed of long polysaccharide chains (glycans) which are covalently bound to a protein core. Proteogylcans also include the polysaccharide chains alone, i.e., without a protein core. Exemplary proteoglycans include hyaluronan (Synvisc), chondroitin sulphate, dermatan sulphate, keratan sulphate, heparan sulphate, heparin, and oligosaccharides. In one embodiment, a proteoglycan administered in combination with a compound that modulates bone formation for treatment of OA or to improve the solubility of the compound that modulates bone formation.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG. See also, Pierce Catalog and Handbook, 1994-1995 (Pierce Chemical Co., Rockford, Ill.). See also, e.g., Kuby, J., Immunology, 3rd Ed., W.H. Freeman & Co., New York (1998). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol :5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” or a “VH” refer to the variable region of an immunoglobulin heavy chain of an antibody, including the heavy chain of an Fv, scFv, or Fab. References to “V_{L}” or a “VL” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

“Biological sample” as used herein is a sample of biological tissue or fluid that contains nucleic acids or polypeptides, e.g., of a Wnt, Fzd, or SFRP protein, polynucleotide or transcript. Such samples include, but are not limited to, tissue isolated from primates, e.g., humans, or rodents, e.g., mice, and rats. Biological samples may also include sections of tissues such as biopsy and autopsy samples, frozen sections taken for histologic purposes, blood, plasma, serum, sputum, stool, tears, mucus, hair, skin, bone cartilage, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff& Henikoff, Proc. Natl. Acad. Sci. USA 89:10915 (1989)) alignments (B) of 50, expectation (E) of 10, M=5, N=4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor & Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically 25 to approximately 500 amino acids long. Typical domains are made up of sections of lesser organization such as stretches of (-sheet and (-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The radioisotope may be, for example, ³H, ¹⁴C, ³²P, ³⁵S, or ¹²⁵I. In some cases, particularly using antibodies against the proteins of the invention, the radioisotopes are used as toxic moieties, as described below. The labels may be incorporated into the nucleic acids, proteins and antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). The lifetime of radiolabeled peptides or radiolabeled antibody compositions may extended by the addition of substances that stablize the radiolabeled peptide or antibody and protect it from degradation. Any substance or combination of substances that stablize the radiolabeled peptide or antibody may be used including those substances disclosed in U.S. Pat. No. 5,961,955.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effectoer” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. By “specifically bind” herein is meant that the antibodies bind to the protein with a K_{D} of at least about 0.1 mM, more usually at least about 1 μM, preferably at least about 0.1 μM or better, and most preferably, 0.01 μM or better.

Specific binding to an antibody under such conditions requires an antibody that is selected for its specificity for a particular protein. For example, polyclonal antibodies raised to a particular protein, polymorphic variants, alleles, orthologs, and conservatively modified variants, or splice variants, or portions thereof, can be selected to obtain only those polyclonal antibodies that are specifically immunoreactive with Wnt or Frizzled proteins and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity).

As used herein “small molecule” includes nucleic acids (e.g., RNAi or siRNA), peptides, small organic molecules, or combinations thereof.

### DETAILED DESCRIPTION OF THE INVENTION

Introduction

The present invention is based, at least in part, on the observation that mutations in the FRZB gene are associated with osteoarthritis (OA). The present disclosure provides methods for identifying mutations in FRZB that can predispose individuals to OA or that are more prevalent in patients with OA. The invention also provides methods to detect changes in levels of WNT/FZD pathway member proteins that are associated with OA as well as methods to identify compounds that modulate bone formation and that affect the activity or expression of a WNT/FZD pathway member. Such compounds can be used to treat OA or to lessen the likelihood of developing the disease in an individual with a predisposition to developing OA.

Recently, several groups have shown that polymorphisms in a gene called LDL receptor-related protein 5 (LRP5) control bone density in humans (Boyden, L. M., et al., N Engl J Med., 346:1513-1521 (2002); Little, R. D., et al., Am J Hum Genet., 70:11-19 (2002); Gong, Y., et al., Cell, 107:513-523 (2001)). Patients who inherit two mutations in the LRP5 gene, which eliminate completely the function of the protein, develop the osteoporosis-pseudoglioma syndrome (OPPG) (Gong, Y., et al., Cell, 107:513-523 (2001)). Furthermore, carriers of single LRP5 mutations have reduced bone density compared to control subjects (Boyden, L. M., et al., N Engl J Med., 346:1513-1521 (2002); Van Wesenbeeck, et al., Am J Hum Genet., 72:763-771 (2003)). These findings suggest that other mutations capable of augmenting the function of the LRP5-related signaling pathway might increase bone density around and in the subchondral region juxtaposed to the joint, and hence predispose to OA.

The LRP5 and LRP6 proteins have been shown to bind to, and to enhance, signaling of wnt family proteins through their cognate frizzled (Fzd) receptors (Tamai, K., et al., Nature, 407:530-535 (2000); Wehrli, M., et al., Nature, 407:527-530 (2000)). Wnt signaling through the canonical β-catenin dependent pathway plays an essential role in bone and joint development during embryogenesis, by regulating the production of many genes involved in cell proliferation, differentiation, and matrix production (Shtutman, M., et al., Proc Natl Acad Sci USA, 96:5522-5527 (1999); Wielenga, V. J., et al., Am J Pathol., 154:515-523 (1999); Riddle, R. D. et al., Cell, 83:631-640 (1995); Capdevila, J. et al., Dev Biol., 193:182-194 (1998); Gradl, D. et al., Mol Cell Biol., 19:5576-5587 (1999); He, T. C., et al., Science, 281:1509-1512 (1998)). For example, local overexpression of wntl4 in chicken embryos, using retroviral expression vectors, is sufficient to cause diarthrodial joint formation (Hartmann, C., and C. J. Tabin, Cell, 104:341-351 (2001)). The dysfunctional mutations of LRP5 described above are thought to reduce bone mass by diminishing wnt signaling Gong, Y., et al., Cell, 107:513-523 (2001)). By analogy, mutations that strengthen wnt signaling would be expected to increase bone mass, but only at the specific joints and bones where the relevant gene products are expressed.

There are several known inhibitors of wnt signaling. They include the secreted frizzled-related proteins (sFRPs), the wnt inhibitor factors (WIF), and the Dickkopf proteins (DKK) (Leyns, L., et al., Cell, 88:747-756 (1997); Melkonyan, H. S., et al., Proc Natl Acad Sci USA, 94:13636-13641 (1997); Glinka, A., et al., Nature, 391:357-362 (1998); Semenov, M. V., et al., Curr Biol., 11:951-961 (2001); Hsieh, J. C., et al., Nature, 398:431-436 (1999)). The sFRP3 gene is also known as FRZB, which stands for Frizzled motif associated with Bone development. By binding to wnt molecules, secreted FRZB can inhibit efficient wnt interaction with Fzd receptors. The FRZB gene is expressed mainly in the developing long bones of the human fetus, and in cartilage (Hoang, B., et al., J Biol Chem., 271:26131-26137 (1996); Wada, N., et al., Int J Dev Biol., 43:495-500 (1999)).

The FRZB gene is encoded on human chromosome 2q31. Several studies of familial OA using microsatellite markers have assigned an OA susceptibility gene to the same chromosomal region (Loughlin, J., et al., Rheumatology (Oxford), 39:377-381 (2000); Loughlin, J., et al., Eur J Hum Genet., 10:562-568 (2002); Loughlin, J., et al., Rheumatology (Oxford), 41:955-956 (2002); Wright, G. D., et al., Ann Rheum Dis., 55:317-319 (1996)). By analyzing SNPs from both familial (sib pairs) and sporadic patients with knee and hip OA, two non-conservative mutations in FRZB (arg-trp at position 200, and arg-gly at position 324), were found to occur with more frequency in patients with OA. These mutations also alter the function of the FRZB protein, and thus, alter the activity of the WNT/FZD signaling pathway.

WNT/FZD Signaling Pathways

Soluble Wnt glycoproteins have been demonstrated to transmit signals by binding to the seven transmembrane domain G-protein coupled-receptor frizzled (Bhanot, P. et al. Nature 382:225-230 (1996); Yang-Snyder, J. et al. Curr Biol 6:1302-1306 (1996); Leethanakul, C. et al. Oncogene 19:3220-3224 (2000)). Upon Wnt signaling, a cascade is initiated that results in the accumulation of cytoplasmic beta-catenin and its translocation to the nucleus. In the nucleus beta-catenin binds a specific sequence motif at the N terminus of lymphoid-enhancing factor/T cell factor (LEF/TCF) to generate a transcriptionally active complex (Behrens, J. et al Nature 382:638-642 (1996)). Beta-catenin interacts with multiple other proteins such as cadherin, which it links to the cytoskeleton (Hoschuetzky, H. et al. J Cell Biol 127:1375-1380 (1994); Aberle, H. et al, J Cell Sci 107:3655-3663 (1994)). It also associates with the adenomatous polyposis coli (APC) tumor suppressor protein and glycogen synthetase 3 beta (GSK3β) (Rubinfeld, B. et al, Science 272:1023-1026 (1996)). These proteins function to negatively regulate beta catenin by facilitating phosphorylation near the aminoterminus and thus accelerating its proteolytic degradation (Yost, C. et al, Genes Dev 10:1443-1454 (1996)).

The canonical Wnt/Fzd signaling cascade leads to the accumulation of cytoplasmic β-catenin and its translocation to the nucleus. In the nucleus beta-catenin binds a specific sequence motif at the N terminus of lymphoid-enhancing factor/T cell factor (LEF/TCF) to generate a transcriptionally active complex (Behrens J et al Nature 382, 638-642 (1996)). In one embodiment, activation of Wnt/Fzd signaling pathway is determined using a reporter gene construct, whose expression is dependent on transcriptional activation of LEF/TCF, e.g., TOPFLASH.

Methods to Identify Biomarkers for Osteoarthritis

Products of the wnt, frizzled (fzd), secreted frizzled-related protein (SFRP) and LDL receptor-related protein (LRP) gene families play roles in the development and maintenance of joints and bones. Consequently, polymorphisms of these genes that alter protein expression or function are potential biomarker candidates for osteoarthritis (OA) susceptibility and progression. Some forms of OA are marked by increased bone density in affected joints. (Sowers, M., L., et al., Arthritis Rheum, 42:483-489 (1999); Hannan, M. T., et al., Arthritis Rheum, 36:1671-1680 (1993) Hart, D. J., et al., Ann Rheum Dis., 53:158-162 (1994); Burger, H., et al., Arthritis Rheum, 39:81-86 (1996); Zhang, Y., et al., J Rheumatol, 27:1032-1037 (2000); Lane, N. E., and M. C. Nevitt, J Rheumatol, 21:1393-1396 (1994); Lane, N. E., et al., J Bone Miner Res., 10:257-263 (1995)). Wnt signaling through the canonical β-catenin dependent pathway plays an essential role in bone and joint development during embryogenesis, by regulating the production of many genes involved in cell proliferation, differentiation, and matrix production (Shtutman, M., et al., Proc Natl Acad Sci USA, 96:5522-5527 (1999); Wielenga, V. J., et al., Am J Pathol., 154:515-523 (1999); Riddle, R. D. et al., Cell, 83:631-640 (1995); Capdevila, J. et al., Dev Biol., 193:182-194 (1998); Gradl, D. et al., Mol Cell Biol., 19:5576-5587 (1999); He, T. C., et al., Science, 281:1509-1512 (1998)). Mutations that strengthen wnt/fzd pathway signaling can increase bone mass at the specific joints and bones where the relevant gene products are expressed, thus, predisposing an individual to OA. In one embodiments mutations include increased or decreased expression of wnt/fzd pathway members. In another embodiment mutations include functional mutations in wnt/fzd pathway members. Mutations in a wnt/fzd pathway member can be used to identify biomarkers that are useful to a predisposition to OA or to diagnose OA in an individual. Mutations in wnt/fzd pathway members can include, for example, loss of function mutations in wnt signaling antagonists (SFRPs, LRPs, WIFs, DKKs), or gain of function mutations in wnt signaling agonists (wnts, Fzds, WISPs). Mutations in a wnt/fzd pathway member, i.e., biomarkers, can be identified by analysis of a wnt/fzd pathway member nucleic acid or by analysis of a wnt/fzd pathway member polypeptide or a downstream wnt/fzd regulated gene product.

Analysis of a wnt/fzd Pathway Member Nucleic Acid

In one aspect, wnt/fzd pathway member genes (or their expression levels) are detected in different patient samples for which either diagnosis or prognosis information is desired. For example, OA is evaluated by a determination of a mutation in a wnt/fzd pathway member gene in the patient. Methods of evaluating the presence and/or copy number of a particular gene or to determine the presence or absence of polymorphism or other mutation in the gene are well known to those of skill in the art. For example, hybridization based assays can be used for these purposes.

Amplification-Based Assays.

In some embodiments, amplification-based assays can be used to analyze wnt/fzd pathway member nucleic acid sequence in a sample. In such amplification-based assays, the nucleic acid sequences act as a template in an amplification reaction (e.g. Polymerase Chain Reaction (PCR). Amplification based assays can be used to identify mutations known to predispose individuals to OA or to determine the copy number of the template nucleic acid present in the sample.

Analysis of wnt/fzd pathway members nucleic acid sequences using PCR techniques can be performed in a number of ways. For example, allele specific PCR primers can be designed that will bind preferentially to control nucleic acid sequences or to mutant sequences. Such primers can be used to screen for mutations in wnt/fzd pathway members. Those of skill will recognize that under certain conditions, the technique can be used to identify wild type wnt/fzd pathway nucleic acids or heterozygous or homozygous mutations in a wnt/fzd pathway nucleic acids. PCR primers can also be used to amplify nucleic acids surrounding a suspected mutation site. The PCR amplification product can then be directly sequenced to determine the presence of a mutation in a sample.

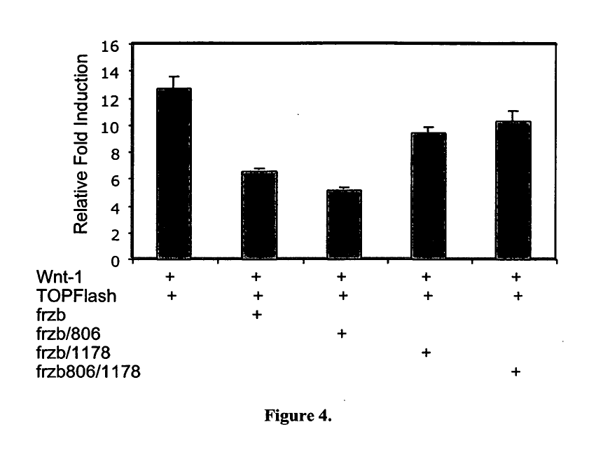

For example, the present application provides mutations in the FRZB gene that are associated with both familial and sporadic osteoarthritis of the hip and knee joint. (See, e.g., example 1 and FIG. 4.) A commonly occurring SNP was present in exon 4, namely a C->T change at position 806 of Genbank reference sequence NM_{—}001463. This SNP was present in both homozygous and heterozygous individuals and would putatively result in an amino acid change from arginine to tryptophan (R200W). A second, non-synonymous polymorphism was found in exon 6 at position 1178. This unusual C to G mutation results in an arginine to glycine substitution (R324G) in the cytoplasmic C-terminal region of the protein.

Allele specific PCR primers can be designed to identify mutants at either position 806 or 1178. For example, the following primers and assay can be used to identify mutants at position 1178:

mutant allele primers

| | |
|---|---|
| (Forward) | gTTAgAATCATggAAATAATgACCCTggTg |
| (Reverse) | TTACTTTTTgTATTTCgggATTTAgTTggC |

wild type allele primers

| | |
|---|---|
| (Forward) | CTggCAggAACTCgAACCCCCggCAAgCAC |
| (Reverse) | CTTAAgAGTCTgCCCCCAAACCATTACAAA |

One ng of genomic DNA is amplified with a cocktail of the four primers at 200 nM each for 35 cycles with 30 sec at 94° C. and 60 sec at 62° C. using a Biorad thermocycler in 96 well format. The products are separated on a 1.5% agarose gel. The product of the wild type allele is 250 bp and the mutant allele amplifies a 207 bp product. Those of skill will recognize that size of the amplification products can be used to determine the genotype of an individual, i.e., homozygous wild type, homozygous mutant, and heterozygous mutant.

FRZB mutations can also be characterized by direct sequence analysis. In the dbSNP national databank there are reports of different mutations at position 806. Sequence analysis identifies the mutations. Previously PCR primers were designed and conditions optimized for the six FRZB exons and the 5′ and 3′ untranslated regions (UTR) (See, e.g., Table 4). The primer sets for exons 4 and 6 will be used for exon 4 and 6 specific mutations. Other primer sets can be used for direct sequencing of mutations in other FRZB exons. After amplification, the double stranded PCR products are sequenced in both directions.

In a quantitative amplification, the amount of amplification product will be proportional to the amount of template in the original sample. Comparison to appropriate (e.g. individuals without OA) controls provides a measure of the copy number.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). The known nucleic acid sequence for the genes is sufficient to enable one of skill to routinely select primers to amplify any portion of the gene.

Real time PCR is another amplification technique that can be used to determine gene copy levels or levels of mRNA expression. (See, e.g., Gibson et al., Genome Research 6:995-1001, 1996; Heid et al., Genome Research 6:986-994, 1996). Real-time PCR is a technique that evaluates the level of PCR product accumulation during amplification. This technique permits quantitative evaluation of mRNA levels in multiple samples. For gene copy levels, total genomic DNA is isolated from a sample. For mRNA levels, mRNA is extracted from tumor and normal tissue and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, β-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Other suitable amplification methods include, but are not limited to ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.

Detection of wnt/fzd Pathway Member Gene Expression

Wnt/fzd pathway member gene expression level can also be assayed as a marker for OA or as a marker for a predisposition to OA. In preferred embodiments, activity of the wnt/fzd pathway member gene is determined by a measure of gene transcript (e.g. mRNA), by a measure of the quantity of translated protein, or by a measure of gene product activity.

Methods of detecting and/or quantifying the gene transcript (mRNA or cDNA) using nucleic acid hybridization techniques are known to those of skill in the art (see Sambrook et al. supra). For example, one method for evaluating the presence, absence, or quantity of mRNA involves a Northern blot transfer.

The probes can be full length or less than the full length of the nucleic acid sequence encoding the protein. Shorter probes are empirically tested for specificity. Preferably nucleic acid probes are 20 bases or longer in length. (See Sambrook et al. for methods of selecting nucleic acid probe sequences for use in nucleic acid hybridization.) Visualization of the hybridized portions allows the qualitative determination of the presence or absence of mRNA.

In another preferred embodiment, a transcript (e.g., mRNA) can be measured using amplification (e.g. PCR) based methods as described above for directly assessing copy number of DNA. In a preferred embodiment, transcript level is assessed by using reverse transcription PCR (RT-PCR). In another preferred embodiment, transcript level is assessed by using real-time PCR.

Hybridization-Based Assays

Hybridization assays can be used to detect copy number of wnt/fzd pathway member genes. Hybridization-based assays include, but are not limited to, traditional “direct probe” methods such as Southern blots or in situ hybridization (e.g., FISH), and “comparative probe” methods such as comparative genomic hybridization (CGH). The methods can be used in a wide variety of formats including, but not limited to substrate- (e.g. membrane or glass) bound methods or array-based approaches as described below.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes are typically labeled, e.g., with radioisotopes or fluorescent reporters. Preferred probes are sufficiently long so as to specifically hybridize with the target nucleic acid(s) under stringent conditions. The preferred size range is from about 200 bp to about 1000 bases.

In some applications it is necessary to block the hybridization capacity of repetitive sequences. Thus, in some embodiments, tRNA, human genomic DNA, or Cot-1 DNA is used to block non-specific hybridization.

In comparative genomic hybridization methods a first collection of (sample) nucleic acids (e.g. from a possible tumor) is labeled with a first label, while a second collection of (control) nucleic acids (e.g. from a healthy cell/tissue) is labeled with a second label. The ratio of hybridization of the nucleic acids is determined by the ratio of the two (first and second) labels binding to each fiber in the array. Where there are chromosomal deletions or multiplications, differences in the ratio of the signals from the two labels will be detected and the ratio will provide a measure of the copy number.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In Situ Hybridization Protocols, Choo, ed., Humana Press, Totowa, N.J. (1994), etc. In one particularly preferred embodiment, the hybridization protocol of Pinkel et al. (1998) Nature Genetics 20: 207-211, or of Kallioniemi (1992) Proc. Natl Acad Sci USA 89:5321-5325 (1992) is used.

A variety of nucleic acid hybridization formats are known to those skilled in the art. For example, common formats include sandwich assays and competition or displacement assays. Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

Typically, labeled signal nucleic acids are used to detect hybridization. The labels may be incorporated by any of a number of means well known to those of skill in the art. Means of attaching labels to nucleic acids include, for example nick translation, or end-labeling by kinasing of the nucleic acid and subsequent attachment (ligation) of a linker joining the sample nucleic acid to a label (e.g., a fluorophore). A wide variety of linkers for the attachment of labels to nucleic acids are also known. In addition, intercalating dyes and fluorescent nucleotides can also be used.

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent labels (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label may be added to the nucleic acids prior to, or after the hybridization. So called “direct labels” are detectable labels that are directly attached to or incorporated into the sample or probe nucleic acids prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

The methods of this invention are particularly well suited to array-based hybridization formats. For a description of one preferred array-based hybridization system see Pinkel et al. (1998) Nature Genetics, 20: 207-211.

Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

The DNA used to prepare the arrays of the invention is not critical. For example the arrays can include genomic DNA, e.g. overlapping clones that provide a high resolution scan of a portion of the genome containing the desired gene, or of the gene itself. Genomic nucleic acids can be obtained from, e.g., HACs, MACs, YACs, BACs, PACs, Pls, cosmids, plasmids, inter-Alu PCR products of genomic clones, restriction digests of genomic clones, cDNA clones, amplification (e.g., PCR) products, and the like.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays.

Analysis of a wnt/fzd Pathway Member Polypeptide or a Downstream wnt/fzd Regulated Gene Product

The expression level of an wnt/fzd pathway member gene can also be detected and/or quantified by detecting or quantifying the expressed wnt/fzd pathway member polypeptide. The polypeptide can be detected and quantified by any of a number of means well known to those of skill in the art.

Any wnt/fzd pathway member protein or downstream regulated product can be used as a serum biomarker for a predisposition to OA, to diagnose OA, or to monitor the progression of OA. The wnt/fzd pathway member protein can include any of the following: 19 known wnt proteins, 9 fzd proteins, SFRPs, WIFs, DKKs, WISPs, and LRPs.

In one embodiment, wnt/fzd pathway member proteins are used as serum biomarkers for OA. OA is a heterogeneous, slowly progressive disease affecting a tissue with low cell proliferation rate. Sera from patients with OA can express a characteristic “proteonomic spectrum,” consisting of increases and decreases in many native and post-translationally modified proteins, which are the product of the fundamental molecular defects that cause the disease. A unique serum biomarker can be present at high concentrations in some OA patients. The Ciphergen SELDI-TOF mass spectrometry protein chip system has been used successfully to identify biomarkers of diseases, including ovarian cancer, and in other malignancies (Li, J., et al., Clin Chem., 48:1296-1304 (2002) Chapman, K., Biochem Soc Trans., 30:82-87 (2002); Ball, G., et al., Bioinformatics, 18:395-404 (2002); Rosty, C., et al., Cancer Res., 62:1868-1875 (2002); Wellmann, A., et al., Int J Mol Med., 9:341-347 (2002); Petricoin, E. F., et al., Lancet, 359:572-577 (2002); Merchant, M., and S. R. Weinberger, Electrophoresis, 21:1164-1177 (2000)). Wnt/fzd pathway member proteins are also used as serum biomarkers for a predisposition to OA, for diagnosis of OA, or as serum markers for increased bone mineral density.

Mass spectrometry, e.g., the SELDI-TOF mass spec (MS) Protein Chip System developed by Ciphergen, can be used to fractionate minute plasma samples via multiple chemistries (anion exchange, cation exchange, hydrophobic interactions, and metal chelation). Moreover, SELDI-TOF MS is a high-throughput protein profiling technique. Such data is analysed with use of bioinformatics tools. Available software packages are used to generate and store proteonomic spectra.

In a representative experiment, sera from OA patients is fractionated using a 70 kDa size exclusion, and anion or cation exchange spin columns, as appropriate. After elution from the column, each fraction is analyzed with the ProteinChip Reader to check for the presence of the target protein, e.g., wnt/lfzd pathway member proteins or downstream wnt/fzd regulated gene products.

The fractions containing the target protein are concentrated by speed-vac and subjected to separation by SDS-PAGE. The proteins are stained by Coomassie Brilliant Blue. The bands that correspond to the molecular weight of the target proteins are excised from the gel. The target protein is identified by peptide mapping, after digestion in gel with trypsin overnight at 37° C. A part of the gel displaying no band (control digest) also is excised and digested with trypsin as a control. The molecular weight of each peptide is measured with the ProteinChip reader (Ciphergen). The molecular weights of peptides derived from the target proteins is analyzed with ProFound software (prowl.rockefeller.edu/cgi-bin/ProFound) or other appropriate software. Sequence information for one or more of the tryptic peptides derived from the target proteins is further analyzed by Post Source Decay (PSD) analysis. The fragmentation pattern of the peptide is gathered by MALDI-TOF MS with PSD function. The sequence is determined by analyzing the patterns with Mascot software (Matrix Science, London, UK). Similar approaches to biomarker identification by serial SELDI-TOF MS identification and tryptic digest protein analysis have been used by (Li, W., et al., J Mol Biol., 323:225-236 (2002); Uchida, T., et al., J Proteome Res., 1:495-499 (2002)).

Both immunoblotting and immunodepletion are used to confirm the identities of the candidate biomarkers identified as described above. For the immunoblotting studies, the partially purified samples separated by SDS-PAGE are transferred to PDF membranes and probed with anti-peptide antibodies that have been prepared against the sequences obtained from the protein digests. For immunodepletion analysis, the various antibodies, or control IgG, are adsorbed onto protein G beads. Representative OA or control sera is incubated overnight at 4° C. with the beads, and the supernatants analyzed on the protein chip arrays. Depletion of the biomarker by the specific antibody, but not the control IgG provides support for the peak identification.

Other methods of detecting and quantifying wnt/fzd pathway member polypeptides may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like. Immunohistochemical methods can also be used to detect wnt/fzd pathway member protein. With immunohistochemical staining techniques, a cell sample is prepared, typically by dehydration and fixation, followed by reaction with labeled antibodies specific for the gene product coupled, where the labels are usually visually detectable, such as enzymatic labels, fluorescent labels, luminescent labels, and the like. A particularly sensitive staining technique suitable for use in the present invention is described by Hsu et al. (1980) Am. J. Clin. Path. 75:734-738. The isolated proteins can also be sequenced according to standard techniques to identify polymorphisms.

The wnt/fzd pathway member polypeptide is detected and/or quantified using any of a number of well recognized immunological binding assays (see, e.g., U.S. Pat. Nos. 4,366,241; 4,376,110; 4,517,288; and 4,837,168). For areview of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (polypeptide or subsequence). The capture agent is a moiety that specifically binds to the analyte. In a preferred embodiment, the capture agent is an antibody that specifically binds a polypeptide. The antibody (anti-peptide) may be produced by any of a number of means well known to those of skill in the art.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled anti-antibody. Alternatively, the labeling agent may be a third moiety, such as another antibody, that specifically binds to the antibody/polypeptide complex.

In one preferred embodiment, the labeling agent is a second human antibody bearing a label. Alternatively, the second antibody may lack a label, but it may, in turn, be bound by a labeled third antibody specific to antibodies of the species from which the second antibody is derived. The second can be modified with a detectable moiety, e.g., as biotin, to which a third labeled molecule can specifically bind, such as enzyme-labeled streptavidin. In some embodiments, Western blot analysis is used to detected and or quantify wnt/fzd pathway member protein.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Wnt/fzd pathway member protein can be detected and/or quantified in cells using immunocytochemical or immunohistochemical methods. IHC (immunohistochemistry) can be performed on paraffin-embedded tumor blocks using a wnt/fzd pathway member-specific antibody. IHC is the method of colormetric or fluorescent detection of archival samples, usually paraffin-embedded, using an antibody that is placed directly on slides cut from the paraffin block. To detect and/or quantify wnt/fzd pathway member protein in, for example tissue culture cells or cells from a subject that are not embedded in paraffin (for example, hematopoetic cells) ICC (immunocytochemistry) can be used. ICC is like IHC but uses fresh, non-paraffin embedded cells plated onto slides and then fixed and stained.

Either polyclonal or monoclonal antibodies may be used in the immunoassays of the invention described herein. Polyclonal antibodies are preferably raised by multiple injections (e.g. subcutaneous or intramuscular injections) of substantially pure polypeptides or antigenic polypeptides into a suitable non-human mammal. The antigenicity of peptides can be determined by conventional techniques to determine the magnitude of the antibody response of an animal that has been immunized with the peptide. Generally, the peptides that are used to raise the anti-peptide antibodies should generally be those which induce production of high titers of antibody with relatively high affinity for the polypeptide.

Preferably, the antibodies produced will be monoclonal antibodies (“mAb's”). For preparation of monoclonal antibodies, immunization of a mouse or rat is preferred. Polyclonal antibodies can also be used.

It is also possible to evaluate an mAb to determine whether it has the same specificity as a mAb of the invention without undue experimentation by determining whether the mAb being tested prevents a mAb of the invention from binding to the subject gene product isolated as described above. If the mAb being tested competes with the mAb of the invention, as shown by a decrease in binding by the mAb of the invention, then it is likely that the two monoclonal antibodies bind to the same or a closely related epitope. Still another way to determine whether a mAb has the specificity of a mAb of the invention is to preincubate the mAb of the invention with an antigen with which it is normally reactive, and determine if the mAb being tested is inhibited in its ability to bind the antigen. If the mAb being tested is inhibited then, in all likelihood, it has the same, or a closely related, epitopic specificity as the mAb of the invention.

The assays of this invention have immediate utility in detecting/predicting the likelihood of OA, and in screening for agents that modulate the subject gene product activity, and in screening for agents that modify bone formation.

Methods of Screening for WNT/FZD Signaling Pathway Function

Assays for wnt/fzd pathway member function can be designed to detect and/or quantify any effect that is indirectly or directly under the influence of the wnt/fzd pathway member protein or nucleic acid, e.g., a functional, physical, or chemical effect. Such assays can be used to test whether a biological sample comprises a functional wnt/fzd pathway member protein, to test whether variant wnt/fzd pathway member polypeptides retain function, or to identify compounds that modulate wnt/fzd pathway member activity in cells. For example, assays are provided to determine the function of the FRZB protein, e.g., assays of transcriptional activation.

Assays may include those designed to test binding activity to either the SFRP or to the Fzd receptor. These assays are particularly useful in identifying agents that modulate Wnt/Fzd signaling activity. Virtually any agent can be tested in such an assay. Such agents include, but are not limited to natural or synthetic polypeptides, including Wnt proteins, antibodies, natural or synthetic small organic molecules, and the like.

Other assays useful in the present invention are those designed to test effects on bone formation, i.e., bone formation assays. These assays include transcriptional activation assays performed in cells including chondrocytes, osteoblasts, or osteoclasts, for example. Other bone formation assays include in vitro calvaria assays. The effect of various Wnt antagonists on osteoclastic bone resorption is determined in murine calvarial cultures. Pairs of hemicalvaria from neonatal mice (4-7 days old) are first preincubated for 24 hours in the presence or absence of a test compound so that each treated hemicalvaria had a corresponding untreated control from the same animal. Then, a stimulator of calcium release (e.g., 20 nM 1,25(OH)₂D3) is added and the incubation continued for a further 72 hours. At the end of the incubation period, the calcium content of the medium and that of the calvaria is determined to permit calculation of the amount of calcium released from the calvaria as a percentage of the its calcium content. (See, e.g., J Bone Miner Res. 9(5):745-51 (1994)).

A modification of the calvarial assay is used to assess the possible interference of the wnt pathways or pathway modulators with bone mineralization in vitro. Calvaria are cultured in a manner similar to that described above but with the following changes: calcium incorporation is stimulated by replacing the 1,25(OH)₂D3 with 2 mM calcium 1,2-glycerophosphate, and the incubation period is reduced to 48 hours. The 24 hour preincubation with bisphosphonate remains unchanged. The amount of calcium and inorganic phosphate incorporated into the calvaria is calculated as the percent increase relative to the initial calcium content. (See, e.g., J Bone Miner Res. 9(5):745-51 (1994)).

Assays may include those designed to test the ability of test agents to bind the wnt/fzd pathway member protein and thereby modulate its activity. Virtually any agent can be tested in such an assay. Such agents include, but are not limited to antibodies, natural or synthetic nucleic acids, natural or synthetic polypeptides, natural or synthetic lipids, natural or synthetic small organic molecules, and the like.

Proteins interacting with the peptide or with the protein encoded by the cDNA (e.g., wnt/fzd pathway member) can be isolated using a yeast two-hybrid system, mammalian two hybrid system, or phage display screen, etc. Targets so identified can be further used as bait in these assays to identify additional proteins that interact with wnt/fzd pathway member or are downstream of wnt/fzd pathway member, which proteins are also targets for drug development (see, e.g., Fields et al., Nature 340:245 (1989); Vasavada et al., Proc. Nat'l Acad. Sci. USA 88:10686 (1991); Fearon et al., Proc. Nat'l Acad. Sci. USA 89:7958 (1992); Dang et al., Mol. Cell. Biol. 11:954 (1991); Chien et al., Proc. Nat'l Acad. Sci. USA 9578 (1991); and U.S. Pat. Nos. 5,283,173, 5,667,973, 5,468,614, 5,525,490, and 5,637,463).

Any of the assays for detecting wnt/fzd pathway member binding are amenable to high throughput screening. High throughput assays for the presence, absence, or quantification of particular nucleic acids or protein products are well known to those of skill in the art. Similarly, binding assays and reporter gene assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Kits Used in Diagnostic, Research, and Therapeutic Applications

For use in diagnostic, research, and therapeutic applications disclosed here, kits are also provided by the invention. In the diagnostic and research applications such kits may include any or all of the following: assay reagents, buffers, wnt/fzd pathway member-specific nucleic acids or antibodies, hybridization probes and/or primers, and the like. For example, a kit could include hybridization probes or primers specific for the FRZB Arg200Trp and/or Arg324Gly mutations. A therapeutic product may include sterile saline or another pharmaceutically acceptable emulsion and suspension base.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of wnt/fzd pathway members, e.g., FRZB. Such kits can be prepared from readily available materials and reagents. For example, such kits can comprise one or more of the following materials: an wnt/fzd pathway member polypeptide or polynucleotide, reaction tubes, and instructions for testing the desired wnt/fzd pathway member function.

A wide variety of kits and components can be prepared according to the present invention, depending upon the intended user of the kit and the particular needs of the user. Diagnosis would typically involve evaluation of a plurality of genes or gene products. The target genes or gene products will be selected based on correlations with important parameters in disease which may be identified in historical or outcome data.

Therapeutic Methods and Administration of Modulators of WNT/FZD Pathway Members

As noted above, modulators of the invention can be used to treat OA and other diseases associated with pathological bone formation. The compounds that inhibit wnt/fzd pathway member activity can be administered by a variety of methods including, but not limited to parenteral (e.g., intravenous, intramuscular, intradermal, intraperitoneal, and subcutaneous routes), topical, oral, local, or transdermal administration. These methods can be used for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges.

The compositions for administration will commonly comprise a modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent to those skilled in the art and are described in more detail in such publications as Remington's Pharmaceutical Science, 15th ed., Mack Publishing Company, Easton, Pa. (1980).

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that antibodies when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions containing modulators of the invention (e.g., functional SFRP proteins, antibodies, or small molecules) can be administered for therapeutic or prophylactic treatments. In therapeutic applications, compositions are administered to a patient suffering from a disease (e.g., osteoarthritis) in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the agents of this invention to effectively treat the patient. An amount of an agonist that is capable of preventing or slowing the development of osteoarthritis in a patient is referred to as a “prophylactically effective dose.” The particular dose required for a prophylactic treatment will depend upon the medical condition and history of the patient, the particular disease being prevented, as well as other factors such as age, weight, gender, administration route, efficiency, etc. Such prophylactic treatments may be used, e.g., in a patient who is suspected of having a significant likelihood of developing osteoarthritis.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

Other known therapies for treating osteoarthritis can be used in combination with the methods of the invention. For example, a modulator of the invention can be administered in combination with glucosamine.

The materials, methods, and devices of the present invention are further illustrated by the examples which follow. These examples are offered to illustrate, but not to limit, the claimed invention.

### EXAMPLES

### Example 1

### Association Between FRZB Mutations and Osteoarthritis

1. Susceptibility Locus for OA on Chromosome 2q

Twin studies have suggested that OA has a strong genetic component (Spector, T. D., et al., Bmj., 312:940-943 (1996); MacGregor, A. J., et al., Arthritis Rheum., 43:2410-2416 (2000); Kaprio, J., et al., Bmj., 313:232 (1996); Sambrook, P. N., et al., Arthritis Rheum., 42:366-372 (1999)). Chromosome 2q has been suggested as a possible locus (Wright, G. D., et al., Ann Rheum Dis., 55:317-319 (1996); Loughlin, J., Rheum Dis Clin North Am., 28:95-109 (2002)). Initially a significant association was described between nodal osteoarthritis (NOA) and two loci on the short arm of chromosome 2 (2q 23-35) using microsatellite marker screening of genomic DNA from 66 sib pairs (Wright, G. D., et al., Ann Rheum Dis., 55:317-319 (1996)). A subsequent analysis of chromosome 2q for linkage to idiopathic OA used a cohort of 481 families that each contained at least one affected sibling pair with severe end-stage OA (defined as having hip or knee joint replacement surgery). This linkage analysis of chromosome 2q using 16 polymorphic microsatellite markers revealed a potential susceptibility locus at 2q31 with a an estimated multipoint logarithm of the odds score (MLS) of 1.22 (Loughlin, J., et al., Rheumatology (Oxford), 39:377-381 (2000)).

2. FRZB (sFRP3) Gene Expression

Candidate genes for OA on chromosome 2q31 include: fibronectin, a glycoprotein present in the extracellular matrix of normal cartilage; the alpha 2 chain of collagen type V, a major constituent of bone; and the interleukin-8 receptor, important in the regulation of neutrophil activation and chemotaxis. In this region is also the gene for FRZB. The FRZB protein was originally discovered to be expressed in embryonic articular cartilage (Hoang, B., et al., J Biol Chem., 271:26131-26137 (1996); Wada, N., et al., Int J Dev Biol., 43:495-500 (1999)). In human embryonic sections, FRZB was prominently found around the chondrifying bone primordia and subsequently in the chondrocytes of the epiphyses (Hoang, B., et al., J Biol Chem., 271:26131-26137 (1996)). This expression in the cartilaginous cores of developing long bones during embryonic and fetal development (6-13 weeks) and homology to the polarity-determining gene frizzled, suggested that FRZB was likely to be involved in the morphogenesis of the mammalian skeleton.

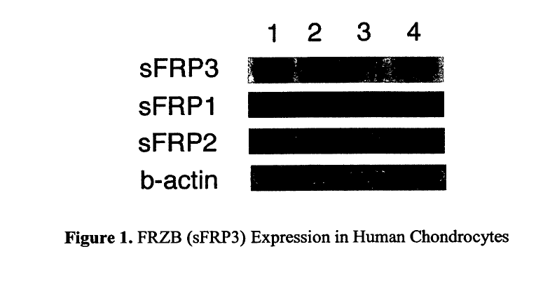

To verify the expression of FRZB in adult cells, lysates of cultured chondrocytes and synovial fibroblast-like cells were tested by immunoblotting (FIG. 1). In the example shown, the chondrocyte lines expressed SFRP-1 and 3 (FRZB), but only weakly expressed SFRP-2. In contrast the synoviocyte lines did express SFRP-1, but not SFRP-3. Thus, there may be differential expression of the SFRP family members amongst varying cell types within the joint itself.

3. FRZB Mutations in Familial OA

To evaluate the potential association of FZRB with OA, the FRZB genes from patients with OA were sequenced. All six FRZB exons were sequenced to identify potential functional mutations as well as single nucleotide polymorphisms (SNPs) as markers. The first samples we screened were from patients with OA and other family members. The DNA from 29 individuals from seven families, with anonymous identifiers, was sequenced to identify all single nucleotide polymorphisms (SNPs) in the FRZB gene.

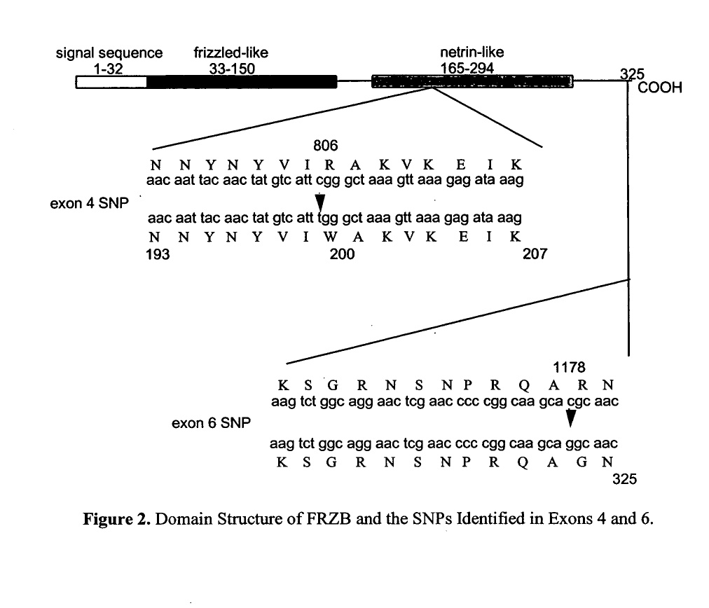

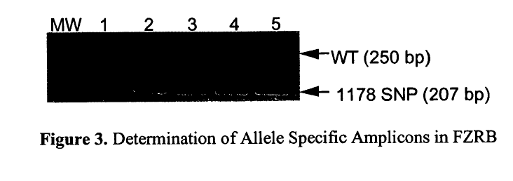

Several SNPs were identified in the FZRB gene in these families (Table 1, FIG. 2). A commonly occurring SNP was present in exon 4, namely a C->T change at position 806 of Genbank reference sequence NM_{—}001463. This SNP was present in both homozygous and heterozygous individuals and would putatively result in an amino acid change from arginine to tryptophan (R200W). A second, non-synonymous polymorphism was found in exon 6. This unusual C to G mutation would result in an arginine to glycine substitution (R324G) in the cytoplasmic C-terminal region of the protein. The affected individuals appeared to be heterozygous for these changes. An allele specific multiplex PCR assay to determine the transmission of this C to G change at the 3′ terminal of exon 6 is shown in FIG. 3.

TABLE 1
Initial SNP analysis of FZRB in Familial OA

| | | | | | Exon 4 | Exon 6 | |
|---|---|---|---|---|---|---|---|
| | Family | OA Diagnosis | 5′UTR 92 | Exon 1 295 | 806 | 1178 | 3′UTR 1397 |
| 1 | A | Hip/Knee | | | C→T | | |
| 2 | A | Hip/Knee | | | | | |
| 3 | B | Normal | | | | | |
| 4 | B | Normal | | | | | |
| 5 | B | Hip | | | | | |
| 6 | B | Hip | | | | | |
| 7 | C | Chondrolysis | | | | C→G | G→A |
| 8 | C | Chondrolysis | | | | C→G | G→A |
| 9 | C | Normal | | | | | |
| 10 | C | Chondrolysis | | | | C→G | G→A |
| 11 | C | Chondrolysis | | | | C→G | G→A |
| 12 | D | Normal | | | C→T* | | |
| 13 | D | Normal | | | C→T* | | |
| 14 | D | Hip | | | C→T | | |
| 15 | D | Normal | | | C→T | | |
| 16 | D | Normal | | | C→T* | | |
| 17 | D | Hip | | | C→T | | |
| 18 | E | Hip | G→A | G→T* | C→T | | |
| 19 | E | Hip | | | C→T | | |
| 20 | F | Hip | | | | | |
| 21 | F | Short stature | | | | | |
| 22 | F | Hip | | | | | |
| 23 | G | Hip | | | C→T | | |
| 24 | G | Hip | | | | | |
| 25 | G | Hip | | | | | |
| 26 | G | Hip | | | C→T | | |
| 27 | G | Hip | | | | | |
| 28 | G | Normal | | | C→T | | |
| 29 | G | Hip | | | | | |

*denotes homozygosity

4. FRZB Mutations in Sporadic OA

A genome wide survey for microsatellite markers was performed using a cohort of 481 OA families that each contained at least one affected sibling pair with severe end-stage disease (ascertained by hip or knee joint replacement surgery). Families with at least two siblings who had undergone one or more replacements of the total hip, or of the total knee, or both, for primary idiopathic OA were recruited for these studies. In the original study a linkage association was found with 2q. As discovered earlier, a finer linkage analysis of chromosome 2q using 16 polymorphic microsatellite markers at an average spacing of one marker every 8.5 cM provided suggestive evidence for a locus at 2q31 with an MLS of 1.22, which increased to 2.19 in those families concordant for hip-only disease (n=31 1) (Loughlin, J., et al., Rheumatology (Oxford), 39:377-381 (2000)). Analysis for the SNPs at positions 1178 and 806 of these previously characterized sib/pairs with OA was performed. As shown in Table 2, significant association with the SNP at position 1178 was found in the female patients from families with hip OA (P=0.03). Because the analysis was confined to familial OA with an established linkage to genes on 2q31, these results could not be generalized to sporadic OA.

Therefore, the FRZB SNP analyses were repeated using a separate cohort of female patients with sporadic hip OA undergoing joint replacement surgery, and the data was compared to female controls. The results confirmed the association of the 1178 SNP with hip OA (p=0.04). Thus, two independent studies with separate cohorts yielded the same conclusion. Moreover, the haplotype analysis of the female hip OA patients versus the female controls revealed that the 806-1178 double mutant haplotype was present at a greatly elevated frequency in the affected patients (27 cases versus 3 controls, 2.5% versus 0.4%, p≦0.002). (See e.g., Table 3).

TABLE 2
Genotype Analysis at Position 1178 of FRZB in
Patients with Hip OA and Controls

| | GENOTYPE | | | | |
|---|---|---|---|---|---|
| 1178 | number (percent) | | | | |
| STRATA | (++) | (+−) | (−−) | P | P* |
| Female hip probands from | 174 | 43 | 2 | 0.03 | 0.02 |
| families with hip OA | (79.5) | (19.6) | (0.9) | | |
| Male hip probands from | 134 | 20 | 1 | 0.95 | 0.90 |
| families with hip OA | (86.5) | (12.9) | (0.6) | | |
| Females with sporadic hip OA | 272 | 54 | 7 | 0.13 | 0.06 |
| | (81.7) | (16.2) | (2.1) | | |
| Males with sporadic hip OA | 194 | 21 | 0 | — | 0.14 |
| | (90.2) | (9.8) | (0.0) | | |
| All females with hip OA | 446 | 97 | 9 | 0.04 | 0.015 |
| | (80.8) | (17.6) | (1.6) | | |
| All males with hip OA | 328 | 41 | 1 | 0.33 | 0.26 |
| | (88.6) | (11.1) | (0.3) | | |
| Female controls | 344 | 46 | 5 | — | — |
| | (87.1) | (11.6) | (1.3) | | |
| Male controls | 308 | 49 | 3 | — | — |
| | (85.6) | (13.6) | (0.8) | | |

*(+−) and (−−) combined

TABLE 3
Haplotype Analysis - Female Patients with
Hip OA versus Female Controls

| HAPLOTYPE | FEMALE HIP | | FEMALE CONTROLS | |
|---|---|---|---|---|
| 806 1178 | Number | % | Number | % |
| Wt—Wt | 862 | 78.5 | 647 | 82.1 |
| Wt - Mut | 88 | 8.0 | 52 | 6.6 |
| Mut - Wt | 121 | 11.0 | 86 | 10.9 |
| Mut—Mut | 27 | 2.5 | 3 | 0.4 |

P = 0.002

5. Association of FRZB Mutations with Predisposition to OA

The FRZB mutations can be biomarkers for increased hip and knee subchondral bone density, which in turn can predispose to OA in women. FRZB haplotypes are determined in a large female population with known bone densities.

The association of hip OA and increased bone mineral density (BMD) was previously described in the SOF patient population in a cross-sectional study (Nevitt, M. C., et al., Arthritis Rheum., 38:907-916 (1995)). Thus, pelvis radiographs from 4,855 subjects were assessed for individual radiographic features of hip OA, including osteophytes, joint space narrowing, subchondral sclerosis, cysts, and femoral head deformity. Hips were graded on a summary scale of 0 (no OA) to 4 (severe OA) based on the number of radiographic features present. Appendicular BMD was measured in all subjects, and hip and spine BMD in 84% of the group. Linear regression was used to examine the association of BMD with hip OA, and to adjust for age, weight, and other determinants of bone mass. Three hundred fifty-one women (7.2%) had mild (grade 2) and 228 (4.7%) had moderate to severe (grade 3-4) radiographic evidence of hip OA. Women with grade 3-4 hip OA had a higher age-adjusted BMD at the femoral neck and Ward's triangle (9-10%; P<0.0001), trochanter (4%; P<0.01), lumbar spine (8%; P<0.0001), and distal radius and calcaneus (5%; P<0.0001 [for each comparison]) compared with those with grade 0-10A in the worse hip. Elevations in BMD were greatest in the femoral neck of hips with OA, in women with bilateral hip OA, and in women with hip osteophytes. These findings were essentially unchanged by adjustment for determinants of bone mass. Hence, elderly Caucasian women with moderate to severe radiographic hip OA had higher BMD in the hip, spine, and appendicular skeleton than did women without hip OA. (Nevitt, M. C., et al., Arthritis Rheum., 38:907-916 (1995)).

FRZB haplotype analysis and mutation genotype of surgical patients who underwent total hip replacement for hip OA and controls was described above. Patients with at least one other family member who was identified as having hip OA, and patients who had sporadic hip OA were studied. In a Caucasian population there was an association between mutations in FRZB at position 1178 and hip OA in women and there was an increase in the number of patients with the 806/1178 double mutant haplotype.

To identify patients that have the 1178/G allele, DNA samples are screened by PCR for size discriminating amplicons as demonstrated above. Allele specific PCR is performed by using a multiplex approach with the following 4 primers:

mutant allele primers

| | |
|---|---|
| (Forward) | gTTAgAATCATggAAATAATgACCCTggTg |
| (Reverse) | TTACTTTTTgTATTTCgggATTTAgTTggC |

wild type allele primers

| | |
|---|---|
| (Forward) | CTggCAggAACTCgAACCCCCggCAAgCAC |
| (Reverse) | CTTAAgAGTCTgCCCCCAAACCATTACAAA |

One ng of genomic DNA is amplified with a cocktail of the four primers at 200 nM each for 35 cycles with 30 sec at 94° C. and 60 sec at 62° C. using a Biorad thermocycler in 96 well format. The products are separated on a 1.5% agarose gel. The product of the wild type allele is 250 bp and the mutant allele amplifies a 207 bp product.

The patients that have the 1178 G allele (mutant) are further characterized for the allele at 806 by sequencing and the 1178 mutation is also be confirmed by direct sequence analysis. In the dbSNP national databank there are reports of different mutations at position 806. A sequence analysis defines the mutations at these positions. Previously PCR primers were designed and conditions optimized for the six FRZB exons and the 5′ and 3′ untranslated regions (UTR) (Table 4). The primer sets for exons 4 and 6 will be used for exon 4 and 6 specific mutations. Other primer sets can be used for direct sequencing of mutations in other FRZB exons. One ng of DNA is amplified with the primers below for 35 cycles of 94° C. 30 sec, 56° C. 60 sec and 72° C. 60 sec. A final extension is done for 10 min at 72° C. The excess primers and nucleotides are removed from 16 ul of the PCR reaction using exonuclease I and shrimp alkaline phosphatase (ExoSAP-IT, Amersham Pharmacia Biotech). Double stranded PCR products are sequenced in both directions with the ABI PRISM® Big Dye™ Terminator Cycle Sequencing Ready Reaction Kit. Data assembly and analysis are performed using SEQUENCER 3.1 software.

TABLE 4

Primer sequences used to amplify the untranslated
regions and the 6 exons of FRZB

| | | |
|---|---|---|
| EXON | Forward | Reverse |

| | | |
|---|---|---|
| 5″ UTR | gggAggTAggAAAgTgCAg | ggTggTTgggCATCTTAgTC |
| Exon 1 | gAggAgAAgCTCCCAgATCC | CCAAgAATTgAggAggCTgT |
| Exon 2 | gCCgACCTCATgACgTTAAT | gACCACAAATgAAAACCAgg |
| Exon 3 | AAgAgCCTTCTCACCACCAA | AAATggCTCCACTCgTTTACA |
| Exon 4 | CCATTCgTTgAATATCAAgTgg | AATgCgAgggTAgCATCTCT |
| Exon 5 | TTCTTgTTCAAgTACTgTTTCCTT | TTCAATgACAgATggCTggT |
| Exon 6/3″ UTR | CCTggTgATATgTgCTTgTgA | CAATTggggTgCAgAAAgTAA |

A genetic association was found between hip OA in women and mutations in a soluble antagonist to wnt signaling, namely FRZB. These genetic mutations result in a FRZB protein with impaired function as assayed in transfection experiments (infra). In the SOF cohort represents a second cohort of patients with defined hip OA for determination of FRZB genotype. The association of hip OA and increased bone density (BMD) was previously described in the SOF patient population in a cross-sectional study. As the LRP5 surface coreceptor of the wnt/frizzled signaling pathway has been linked to bone density, increased bone density may result from impaired inhibition of the wnt/frizzled signaling.

Hip bone mineral density in the OA cases with polymorphisms of FRZB is compared to OA cases without the FRZB polymorphism, and controls without OA will be assessed from DXA measurements obtained at baseline or visit 2 of the SOF study. Bone mineral density is assessed at the femoral neck and total hip. Previous work by Lane and Nevitt have demonstrated that individuals with hip OA characterized by osteophytes or trophic cases have increased bone mineral density at the hip and lumbar spine between 5-9% compared to controls without OA and OA cases without osteophytes.

Subchondral bone mass is assessed by radiographic methods. Subchondral bone thickening is interpreted as subchondral sclerosis by hip radiograph. The presence of subchondral sclerosis by hip radiograph has been assessed. The presence and possibly severity of subchondral sclerosis in OA cases with the FRZB polymorphism and OA cases without the polymorphism is determined. In addition, earlier studies by Nevitt and Lane found the presence of osteophytes on hip radiograph in hip OA cases was significantly associated with increased lumbar spine and hip bone mineral density compared to hip OA cases without osteophytes (Nevitt, M. C., et al., Arthritis Rheum., 38:907-916 (1995)). Therefore, the hip OA cases with a FRZB polymorphism are also assessed for presence and severity of osteophytes compared to those hip OA cases without the polymorphism and controls without OA.

Methods for scoring hip radiographs are known. All hip radiographs from the SOF population have been scored by validated methods that have been previously published (Lane, N. E., and M. C. Nevitt, J Rheumatol, 21:1393-1396 (1994); Nevitt, M. C., et al., Arthritis Rheum., 38:907-916 (1995); Lane, N. E., et al., Arthritis Rheum., 42:854-860 (1999)).

Pelvic radiographs are analyzed using a semi-quantitative assessment of subchondral bone mass by assessing the presence of subchondral sclerosis present on pelvic radiographs. Lane et al. have shown that their groups reliability in assessing the presence and severity of subchondral sclerosis is high, with a kappa statistic of >67% (Lane, N. E., and M. C. Nevitt, J Rheumatol, 21:1393-1396 (1994)).

### Example 2

### Assays for Function of FRZB in Bone Formation

The protein structure of FRZB comprises two functional domains (FIG. 4). The N-terminal domain has been shown to have considerable (>50%) homology with the wnt receptor, frizzled (Hoang, B., et al., J Biol Chem., 271:26131-26137 (1996)). The C-terminal domain shares homology with several proteins central to skeletal development and tissue remodeling (Banyai, L., and L. Patthy, Protein Sci., 8:1636-1642 (1999)). Experiments comparing the ability of wild type FRZB, and the 806 (arg-trp) and 1178 (arg-gly) variants to antagonize wnt-signaling were performed after transient transfection into HEK293 cells (FIG. 4).

The cells were transfected with (i) a wntl vector to maximize signaling, (ii) the wnt-dependent reporter gene TOPflash, or the inactivated reporter gene FOPflash, kindly provided by Hans Clevers [Utrecht, The Netherlands], (iii) a β-galactosidase expressing plasmid to control for transfection efficiency, and (iv) FRZB wild type or mutant genes cloned into pcDNA3 (Invitrogen). Whereas the wild type FRZB vector efficiently inhibited wnt-dependent TOPflash activity, the 1178 mutant, and the 806/1178 double mutant, had diminished activity. Similar experiments are performed in chondrocytes.

FRZB wild type and mutant proteins produced by transfected cells are used to compare the effects of the exogenously added recombinant proteins on the growth and function of human chondrocytes, osteoblasts, and osteoclasts.

### Example 3

### Detection of WNT/FZD Pathway Members in Serum and Association with Osteoarthritis

Unique serum biomarkers can be used to diagnose OA, to individuals with a predeisposition to OA or to asses disease progression. In addition, OA sera can express unique biomarkers or a characteristic “proteonomic spectrum,” consisting of increases and decreases in many native and post-translationally modified proteins, which are the product of the fundamental molecular defects that cause the disease. The Ciphergen SELDI-TOF mass spectrometry protein chip system has been used successfully to identify biomarkers in ovarian cancer, and in other malignancies (Li, J., et al., Clin Chem., 48:1296-1304 (2002) Chapman, K., Biochem Soc Trans., 30:82-87 (2002); Ball, G., et al., Bioinformatics, 18:395-404 (2002); Rosty, C., et al., Cancer Res., 62:1868-1875 (2002); Wellmann, A., et al., Int J Mol Med., 9:341-347 (2002); Petricoin, E. F., et al., Lancet, 359:572-577 (2002); Merchant, M., and S. R. Weinberger, Electrophoresis, 21:1164-1177 (2000)).

Initial studies with the protein chip system were performed with rheumatoid arthritis (RA) synovial fluids. The Biowizard software programidentified 3300-3400 kDa peaks in the RA synovial fluids that were not detectable in OA fluids (Data not shown). The TagIdent program (us.espasy.org/tools/tagident.html) suggested that these biomarkers could be α-defensins. Antibodies to the defensins were already commercially available, and used to immunodeplete the RA synovial fluids, which were then retested. The results confirmed the identity of the peaks as α-defensins.

Serum was analyzed using the Ciphergen protein chip system using essentially the following protocol.

1. Pretreat each surface with the appropriate buffer. Incubate for 10 minutes

| | |
|---|---|
| SAX2 (anion exchange) | PBS, 0.1% Triton pH 7 |
| WCX2 (cation exchange) | 10 mM HCl (5 mins), 1 M NH₄Ac pH 6 |
| IMAC3-Ni²⁺ (metal chelation) | 50 mM NiSO₄, followed by H₂O |
| IMAC3-Cu²⁺ (metal chelation) | 50 mM CuSO₄, followed by H₂O |
| H50 (reverse phase) | 10 ul 50% acetonitrile |

2. 5 ug of serum is added to chip in a volume of 200 ul of binding buffer, in a bioprocessor. Incubate at room temperature for 1-3 hrs.

| | |
|---|---|
| SAX2 | PBS, 0.1% Triton pH 7 |
| WCX2 | 100 mM NH₄Ac, 0.1% Triton pH 6 |
| IMAC3-Ni²⁺ | PBS, 0.1% Triton pH 7 |
| IMAC3-Cu²⁺ | PBS, 0.1% Triton pH 7 |
| H50 (reverse phase) | PBS, 0.1% TFA |

3. Wash the chips in appropriate buffer according to chip surface and stringency desired. Wash each spot three times with 200 ul in the bioprocessor, for 5-10 minutes while shaking.

| Chip Surface | Low Stringency | High Stringency |
|---|---|---|
| SAX2 | PBS, 0.1% Triton pH 7 | 100 mM NaAc, 500 mM NaCl, |
| | | 1% Triton pH 6 |
| WCX2 | 100 mM NH₄Ac, 0.1% | 100 mM Tris-HCl, 0.1% |
| | Triton pH 6 | Triton, pH 9.5 |
| IMAC3-Ni²⁺ | PBS, 0.1% Triton pH 7 | 100 mM NaAc, 500 mM NaCl, |
| | | 1% Triton pH 6 |
| IMAC3-Cu²⁺ | PBS, 0.1% Triton pH 7 | 100 mM NaAc, 500 mM NaCl, |
| | | 1% Triton pH 6 |
| H50 | PBS, 0.1% TFA | PBS, 15% acetonitrile, |
| (reverse | | 0.1% TFA |
| phase) | | |

4. Rinse each chip thoroughly with deionized water. Remove from the bioprocessor for this step. Air dry.

5. Prepare a solution of sinnapinic acid (energy absorbing molecule (EAM)) by adding 800 μl of 50% acetonitrile, 0.5% TFA to 5 mg of the acid. Shake well. Add twice 0.5 μl/spot. Air dry. Store in the dark until reading the chips in the Proteinchip Reader.

6. Machine is calibrated via Ciphergen's All-in-one Protein or Peptide standards.

7. Chips are read twice. Once at low energy (170 nM) for low masses, and a second reading at high energy (220 nM) for high mass determinations.

8. Spectra are normalized, and the BioMarker Wizard program from Ciphergen is used to compare respective spectra. The Tagident program is then used to search for candidate proteins in the database corresponding to the peaks that discriminate between patients and controls.

OA, RA, and normal sera were fractionated on five different types of protein chips. The Biowizard programidentified only four possible biomarkers that distinguished the four OA sera from the RA sera. (Data not shown.) Potential 40 kDa and 44 kDa markers were retained by the cation-exchange chip after a high-stringency wash. The Tagldent program suggested that the markers had molecular masses and isoelectric points consistent with the wnt, WIF, or WISP proteins, i.e., WNT/FZD pathway members. (Data not shown.)

Further anlysis, including peak isolation, fragmentation into peptides, and mass spectrometry fingerprints can be performed to identify biomarkers. The co-identity of the peaks in multiple specimens is confirmed by dot immunoblotting, and by immunodepletion using specific antibodies as described above for the α-defensins.

Insofar as many proteins may be retained simultaneously on the protein chip arrays, it is necessary to enrich potential biomarkers to enable identification. However, devising purification strategies is greatly assisted by the chromatographic affinity information gained during the discovery process. Microspin columns are available corresponding to the anionic and cationic surfaces used on the chip (available through Ciphergen), as well as chips that are capable of molecular weight fractionation. Experiments described previously identified 40 kDa and 44 kDa peaks in OA serum eluted from the weak cation protein chip. Accordingly, spin columns corresponding to the chip surfaces will be used for peak purification. The protein chip system is the screen for the column fractions, allowing rapid monitoring of biomarker elution. Then, the semipurified biomarker is run on a one-dimensional SDS-PAGE gel, excised and exposed to tryptic peptide digestion in situ. The protein chip system is used to analyze the digested peptides, and the masses are compared against protein databases.

Thus, sera from OA patients with the 40 and 44 kDa biomarkers is fractionated using a 70 kDa size exclusion, and cation exchange spin columns. In the former case, samples are diluted 1/5 with 20 uM Tris-HCl, pH8. A 40 uL portion of the diluted sample is added to the column and centrifuged (2000 rpm, 2 min: Fraction 1). Then, a 40 ul portion of the elution buffer (20 mM Tris-HCl, pH 8) is added to the column and centrifuged again (Fraction 2). The last process will be repeated as necessary. Each fraction is analyzed with the ProteinChip Reader to check for the presence of the target protein.

The fractions containing the target protein are concentrated by speed-vac and subjected to separation by SDS-PAGE. The proteins are stained by Coomassie Brilliant Blue. The bands at 40-44 kDa that correspond to the molecular weight of the target proteins are excised from the gel. The target protein is identified by peptide mapping, after digestion in gel with trypsin overnight at 37° C. A part of the gel displaying no band (control digest) also is excised and digested with trypsin as a control. The molecular weight of each peptide is measured with the ProteinChip reader (Ciphergen). The molecular weights of peptides derived from the target proteins are analyzed with ProFound software (prowl.rockefeller.edu/cgi-bin/ProFound). Sequence information for one or more of the tryptic peptides derived from the target proteins is further analyzed by Post Source Decay (PSD) analysis. The fragmentation pattern of the peptide ise gathered by MALDI-TOF MS with PSD function. The sequence is determined by analyzing the patterns with Mascot software (Matrix Science, London, UK). Similar approaches to biomarker identification by serial SELDI-TOF MS identification and tryptic digest protein analysis have been used successfully (see e.g., Li, W., et al., J MolBiol., 323:225-236 (2002); Uchida, T., et al., J Proteome Res., 1:495-499 (2002)).

Both immunoblotting and immunodepletion are used to confirm the identities of the candidate biomarkers identified as described above. For the immunoblotting studies, the partially purified samples separated by SDS-PAGE are transferred to PDF membranes and probed with anti-peptide antibodies that have been prepared against the sequences obtained from the protein digests. For immunodepletion analysis, the various antibodies, or control IgG, are adsorbed onto protein G beads. Representative OA or control sera is incubated overnight at 4° C. with the beads, and the supernatants are analyzed on the protein chip arrays. Depletion of the biomarker by the specific antibody, but not the control IgG provides support for the peak identification, as demonstrated in the identification of defensins in the synovial fluids and sera of RA patients.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method to determine a predisposition to osteoarthritis in a patient, the method comprising
a. obtaining biological sample from the patient;
b. detecting a mutation in wnt/fzd pathway member nucleic acid when compared to a control, wherein the mutation indicates a predisposition to osteaoarthritis.

2. The method of claim 1, wherein the mutation indicates a predisposition to a change in bone mass or bone structure at a region adjacent to a joint.

3. The method of claim 1, wherein the mutation results in increased signaling in a wnt/fzd pathway.

4. The method of claim 1, wherein the patient is an adult.

5. The method of claim 1, wherein the patient is female.

6. The method of claim 2, wherein the change in bone mass or bone structure occurs at a region adjacent to a knee joint or a hip joint.

7. The method of claim 2, wherein the change in bone mass or bone structure is detected using a method selected from the group consisting of X-ray, magnetic resonance imaging (MRI), and ultrasound.

8. The method of claim 1, wherein the wnt/fzd pathway member nucleic acid encodes a protein selected from the group consisting of FrzB.

9. The method of claim 1, wherein the wnt/fzd pathway nucleic acid is a FRZB nucleic acid.

10. The method of claim 9, wherein the FRZB nucleic acid encodes a tryptophan at residue 200.

11. The method of claim 10, wherein the FRZB nucleic acid has a C to T change at base 806.

12. The method of claim 9, wherein the FRZB nucleic acid encodes a glycine at residue 324.

13. The method of claim 12, wherein the FRZB nucleic acid has a C to G change at base 1178.

14. The method of claim 9, wherein the FRZB nucleic acid encodes a tryptophan at residue 200 and a glycine at residue 324.

15. The method of claim 14, wherein the FRZB nucleic acid has a C to T change at residue 806 and a C to G change at residue 1178.

16. A method to determine a predisposition to osteoarthritis in a patient, the method comprising
a. obtaining biological sample from the patient;
b. detecting a difference in a wnt/fzd pathway member protein expression when compared to a control, wherein the difference indicates a predisposition to osteoarthritis.

17. The method of claim 16, wherein the biological sample is from serum.

18. The method of claim 16, wherein the mutation indicates a predisposition to a change in bone mass or bone structure at a region adjacent to a joint.

19. The method of claim 16, wherein the difference results in increased signaling in a wnt/fzd pathway.

20. The method of claim 16, wherein the patient is an adult.

21. The method of claim 16, wherein the patient is female.

22. The method of claim 18, wherein the change in bone mass or bone structure occurs at a region adjacent to a knee joint or a hip joint.

23. The method of claim 18, wherein the change in bone mass or bone structure is detected using a method selected from the group consisting of X-ray, magnetic resonance imaging (MRI), and ultrasound.

24. The method of claim 16, wherein the difference is detected using mass spectroscopy (MS).

25. The method of claim 16, wherein the difference is detected using an immunoassay.

26. A method of identifying a compound that modulates bone formation, the method comprising the steps of
(a) contacting a chondrocyte comprising a target protein or fragment thereof, wherein the target protein is a wnt/fzd pathway member protein,
(b) determining the functional effect of the compound upon a bone formation assay, thereby identifying a compound that modulates bone formation.

27. A method of modulating bone formation in a subject, the method comprising administering a therapeutically effective amount of a compound identified using the method of claim 26.

28. The method of claim 27, wherein the subject is a human.

29. The method of claim 27, wherein the subject has a predisposition to develop osteoarthritis, and wherein the compound prevents or slows development of osteoarthritis.

30. The method of claim 27, wherein the subject has osteoarthritis, and wherein the compound is used to treat osteoarthritis.

31. A primer pair for detecting the sequence of a FRZB nucleic acid, the primer pair consisting of a first primer comprising

| | |
|---|---|
| CTggCAggAACTCgAACCCCCggCAAgCAC, | (SEQ ID NO:1) |

and a second primer comprising

| | |
|---|---|
| CTTAAgAGTCTgCCCCCAAACCATTACAAA. | (SEQ ID NO:2) |

32. A primer pair for detecting the sequence of a FRZB nucleic acid, the primer pair consisting of a first primer comprising

| | |
|---|---|
| gTTAgAATCATggAAATAATgACCCTggTg, | (SEQ ID NO:3) |

and a second primer comprising

| | |
|---|---|
| TTACTTTTTgTATTTCgggATTTAgTTggC. | (SEQ ID NO:4) |
